(19) United States
(12) Patent Application Publication
(10) Pub. No.: US 2023/0084205 A1
(43) Pub. Date: Mar. 16, 2023

(54) TECHNIQUES FOR MENOPAUSE AND HOT FLASH DETECTION AND TREATMENT

(71) Applicant: Oura Health Oy, Oulu (FI)

(72) Inventors: Steve Kent, San Francisco, CA (US); Neta Ada Gotlieb, Albany, CA (US); Gerald Pho, Somerville, MA (US); Mari Karsikas, Oulu (FI); Kirstin Elizabeth Aschbacher, San Francisco, CA (US)

(21) Appl. No.: 17/818,101

(22) Filed: Aug. 8, 2022

Related U.S. Application Data
(60) Provisional application No. 63/242,855, filed on Sep. 10, 2021.

(51) Int. Cl. A61B 5/00; A61B 5/0205; A61B 5/026

(52) U.S. Cl. CPC A61B 5/7275; A61B 5/02055; A61B 5/026; A61B 5/4866; A61B 5/746; A61B 5/748; A61B 5/7435; A61B 5/6802

(57) ABSTRACT

Methods, systems, and devices for menopause detection and treatment are described. A system may be configured to receive physiological data associated with a user from a wearable device, and identify one or more physiological indications of a hot flash experienced by the user based on the physiological data satisfying one or more thresholds. Additionally, the system may be configured to determine a metabolic efficiency metric associated with the user based on the received physiological data, and determine a menopause metric for the user based on the metabolic efficiency metric, where the menopause metric is associated with a relative probability that the user will experience menopausal symptoms. The system may then cause a graphical user interface (GUI) of a user device to display information associated with the identified hot flash and/or one or more messages associated with the menopause metric.

### CROSS REFERENCE

The present application for patent claims the benefit of U.S. Provisional Patent Application No. 63/242,855 by KENT et al., entitled “TECHNIQUES FOR MENOPAUSE AND HOT FLASH DETECTION AND TREATMENT,” filed Sep. 10, 2021, assigned to the assignee thereof, and expressly incorporated by reference herein.

### FIELD OF TECHNOLOGY

The following relates to wearable devices and data processing, including techniques for menopause and hot flash detection and treatment.

### BACKGROUND

Some wearable devices may be configured to collect data from users associated with body temperature and heart rate. For example, some wearable devices may be configured to detect cycles associated with women's health. However, conventional cycle detection techniques implemented by wearable devices are deficient.

### BRIEF DESCRIPTION OF THE DRAWINGS

FIG. 1 illustrates an example of a system that supports techniques for menopause and hot flash detection and treatment in accordance with aspects of the present disclosure.

FIG. 2 illustrates an example of a system that supports techniques for menopause and hot flash detection and treatment in accordance with aspects of the present disclosure.

FIG. 3 illustrates an example of a timing diagram that supports techniques for menopause and hot flash detection and treatment in accordance with aspects of the present disclosure.

FIG. 4 illustrates an example of a timing diagram that supports techniques for menopause and hot flash detection and treatment in accordance with aspects of the present disclosure.

FIG. 5 illustrates an example of a timing diagram that supports techniques for menopause and hot flash detection and treatment in accordance with aspects of the present disclosure.

FIG. 6 illustrates an example of timing diagrams that supports techniques for menopause and hot flash detection and treatment in accordance with aspects of the present disclosure.

FIG. 7 illustrates an example of timing diagrams that supports techniques for menopause and hot flash detection and treatment in accordance with aspects of the present disclosure.

FIG. 8 illustrates an example of timing diagrams that supports techniques for menopause and hot flash detection and treatment in accordance with aspects of the present disclosure.

FIG. 9 illustrates an example of timing diagrams that supports techniques for menopause and hot flash detection and treatment in accordance with aspects of the present disclosure.

FIG. 10 illustrates an example of a graphical user interface (GUI) that supports techniques for menopause and hot flash detection and treatment in accordance with aspects of the present disclosure.

FIG. 11 shows a block diagram of an apparatus that supports techniques for menopause and hot flash detection and treatment in accordance with aspects of the present disclosure.

FIG. 12 shows a block diagram of a wearable application that supports techniques for menopause and hot flash detection and treatment in accordance with aspects of the present disclosure.

FIG. 13 shows a diagram of a system including a device that supports techniques for menopause and hot flash detection and treatment in accordance with aspects of the present disclosure.

FIGS. 14 through 18 show flowcharts illustrating methods that support techniques for menopause and hot flash detection and treatment in accordance with aspects of the present disclosure.

### DETAILED DESCRIPTION

Some wearable devices may be configured to collect physiological data from users, including temperature data, heart rate, and heart rate variability (HRV) data, sleep data, respiratory data, and the like. Acquired physiological data may be used to analyze behavioral and physiological characteristics associated with the user, such as movement, menstrual cycles, pregnancy, physiological arousal, and the like. Many users have a desire for more insight regarding their physical health, including their sleeping patterns, activity, and overall physical well-being. In particular, many users may have a desire for more insight regarding women's health, including their menstrual cycle, ovulation, fertility patterns, pregnancy, postpartum, and menopause. However, typical cycle tracking or women's health devices and applications lack the ability to provide robust prediction and insight for several reasons.

First, few biomarkers indicate when a woman is beginning to transition to perimenopause or menopause phase. Second, even for devices that are wearable or that measure a user's biomarkers, typical devices and applications lack the ability to collect other physiological, behavioral, or contextual inputs from the user that can be combined with the measured temperature to more comprehensively understand the complete set of physiological contributors to a woman's cycle.

Aspects of the present disclosure are directed to techniques for detecting hot flashes and methods for menopause, perimenopause, and related symptoms detection and predictions. In particular, computing devices of the present disclosure may receive physiological data from the wearable device associated with the user. Aspects of the present disclosure may identify one or more physiological indications of a hot flash experienced by the user based on the physiological data satisfying one or more thresholds. In some examples, aspects of the present disclosure may determine a metabolic efficiency metric associated with the user based on the received physiological data. As such, aspects of the present disclosure may determine a menopause metric for the user based on the metabolic efficiency metric, where the menopause metric may be associated with a relative probability that the user will experience menopausal symptoms. In some cases, the menopause metric may be associated with a relative probability that the user will experience premenopausal, perimenopausal, menopausal, or postmenopausal symptoms. The menopause metric may indicate where the user is in a timeline of experiencing symptoms associated with menopause. For example, the menopause metric may indicate whether the user is in premenopause, perimenopause, menopause, or postmenopause.

For the purposes of the present disclosure, the term “menopausal symptoms,” “perimenopausal symptoms,” “hot flash,” and like terms, may be used to refer to an onset of symptoms associated with a user's cycle including a series of natural changes in hormone production as the user transitions to perimenopausal or menopausal phase. Perimenopause and menopause may start during the user's 30's and 40's and last for 7 to 15 years. Perimenopause and menopause may indicate a decline in reproductive hormones and may be signaled by 12 months since a last menstruation.

During the transition into menopause, many women suffer from significant sleep disruption due to hot flashes, for example, as well as increases in depression and anxiety, and other symptoms that decrease quality of life and also mark an increased risk for cardiovascular disease (e.g., sleep disturbances, hot flashes, vaginal dryness, brain fog, fatigue, sweating, hair loss, irritability, anxiety). In some cases, detecting and treating perimenopausal symptoms (e.g., hot flashes) at an early stage may reduce later-life health risks for women, specifically risks for cardiovascular disease and cognitive dysfunction. In such cases, techniques to detect and intervene for women experiencing hot flashes, in order to improve quality of life, sleep, and mood, and to reduce future health risks may be desired. For example, methods and techniques to help women understand in a personalized way how to optimize lifestyle changes to reduce perimenopausal symptoms may be desired.

Hot flashes may be an example of a perimenopausal and menopausal symptom. In some cases, hot flashes may be related to the hormonal changes before, during, and after menopause that involve increased sympathetic activation. The frequency and intensity of hot flashes may vary among women, although women may experience hot flashes according to a circadian rhythm, peaking at the evening time. Hot flashes in the first half of the night may lead to awakenings and arousals, however, during the second half of the night the hot flashes may be subsided when rapid eye movement (REM) sleep suppresses thermoregulatory effector responses. Hot flashes can be measured objectively, using sternal skin conductance, an electrical measure of sweating. During a hot flash, a user may experience a sudden feeling of warmth spreading through their chest, neck, and face, a flushed appearance with red, blotchy skin, rapid heartbeat, perspiration (e.g., on the upper body), increased breath rate, a chilled feeling as the hot flash diminishes, and anxiety. In some cases, a user may experience a hot flash for a minute or up to five minutes. During a hot flash, the blood rush to the blood vessels nearest to the skin may raise skin temperature by five to seven degrees Celsius.

For example, hot flashes may be triggered by elevations in core body temperature within a reduced thermoneutral zone. For example, hot flashes may be associated with a narrowed thermoregulatory zone. In such cases, the system may be able to detect a narrowed thermoregulatory zone in order to provide metrics that may enable women to understand how behavior changes (e.g., improvements in sleep, exercise, diet, and mood) may help reduce their hot flashes or other perimenopausal and/or menopausal symptoms.

Some aspects of the present disclosure are directed to the detection of menopause before the user experiences symptoms and effects of menopause. However, techniques described herein may also be used to detect an onset of menopause in cases where the user does not become symptomatic, or does not become aware of their symptoms. In some implementations, the computing devices may be able to detect the onset of menopause using a temperature sensor and/or heart rate sensor. In such cases, the computing devices may estimate the retrospective dates of menopause onset without the user tagging or labeling these events.

In some cases, the system may provide a metric to quantify the efficiency of thermoregulation that may be associated with hot flash frequency, hot flash severity, women's age, or cycle parameters. In such cases, the system may be able to provide this data metric to women as a metric to help them optimize a behavioral approach to treating and reducing the frequency or severity of hot flashes during the perimenopausal or menopausal phase. In some cases, the system may provide a metric to quantify the efficacy of hormone replacement therapy (HRT). In such cases, the system may be able to provide this metric to the user as a metric to help them organize a behavioral approach to treating and reducing symptoms or effects associated with perimenopause or menopause.

Sleep disturbance may be a symptom associated with perimenopause and menopause. Sleep disturbance may be caused by multiple factors both internal and external to the body. In some cases, users who have started HRT may see improvements in subjective sleep patterns. The system may be used to monitor improvement in the quantity and quality of sleep during and following the commencement of HRT and provide feedback to users. Similarly, the system described herein may monitor a user's sleep data (e.g., sleep latency, duration/proportion of sleep within respective sleep stages, etc.) to detect hot flashes, determine a metabolic efficiency of users, and the like.

In some cases, the overall health of the user may be associated with higher HRV reflecting the intrinsic flexibility within a physiological system to deal with acute stress and changes in the environment. In some examples, the overall health of the user may improve with better sleep. Lower HRV may be associated with a system that lacks the capacity to vary its response to such change. For example, HRV may decrease as the user ages and may be observed to decrease post-menopause. In some cases, decreases in HRV may also be also seen in younger women with premature ovarian failure, early menopause, and oophorectomy (surgical menopause), highlighting the physiological effect of sex hormones, and more specifically oestradiol in modifying HRV. The system may be used to monitor improvement in HRV during and following the commencement of HRT and provide feedback to users.

Techniques described herein may notify a user of the detected/predicted menopausal symptoms in a variety of ways. For example, a system may cause a graphical user interface (GUI) of a user device to display a message or other notification to notify the user of a predicted future hot flash, and make recommendations to the user. In one example, the GUI may display a time interval that the future hot flash (e.g., menopausal symptom) is predicted to occur and recommendations that the user prepare for the perimenopausal or menopausal symptoms based on previously input symptoms. For example, the system may generate recommendations for users about when the user might consider avoiding certain foods and/or drinks, intensifying the user's training, or building in more recovery time based on the predicted perimenopausal or menopausal symptoms.

A GUI may also include graphics/text that indicate the data used to make the detection/prediction of upcoming perimenopausal or menopausal symptoms. In some cases, the message or notification may be derived from the statistical likelihood that a given tag or physiological pattern may be associated with a “hot flash” tag. The system may also transmit a message to the user to confirm or correct the predicted perimenopausal or menopausal symptoms in order to strengthen the system's ability to provide the user with accurate insights, recommendations, or predictions. Based on the early warnings (e.g., before noticeable symptoms), a user may take early steps that may help reduce the severity of upcoming symptoms associated with menopause. Additionally, a user may modify/schedule their daily activities (e.g., work and leisure time) based on the early warnings and estimated future perimenopausal or menopausal symptoms. A GUI may also include graphics/text that reflects physiological changes associated with HRT, such as improvement in sleep and HRV metrics.

Aspects of the disclosure are initially described in the context of systems supporting physiological data collection from users via wearable devices. Additional aspects of the disclosure are described in the context of example timing diagrams and an example GUI. Aspects of the disclosure are further illustrated by and described with reference to apparatus diagrams, system diagrams, and flowcharts that relate to techniques for menopause and hot flash detection and treatment.

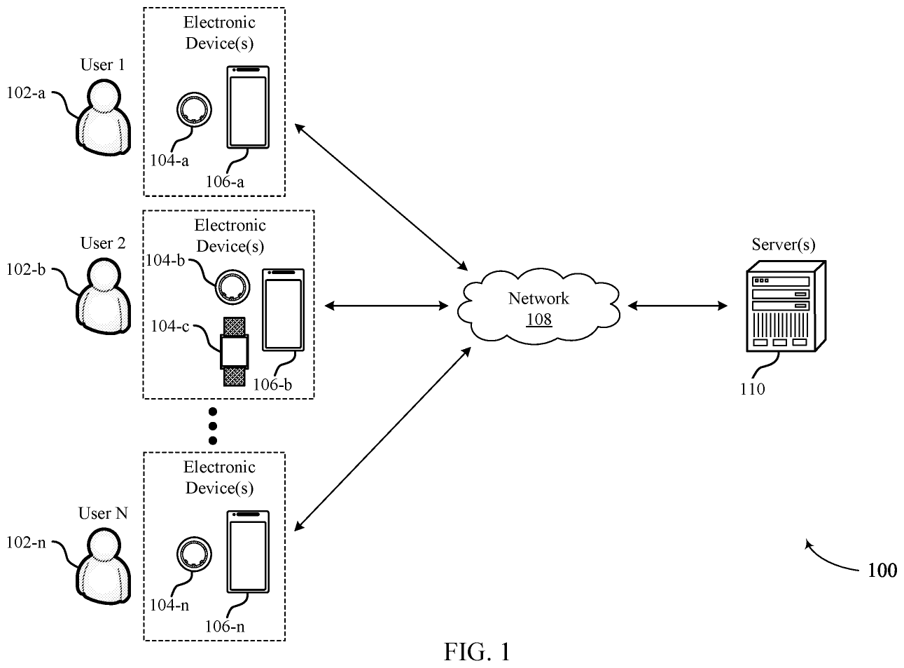

FIG. 1 illustrates an example of a system 100 that supports techniques for menopause and hot flash detection and treatment in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, HRV, actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more LEDs (e.g., red LEDs, green LEDs) that emit light on the palm-side of a user's finger to collect physiological data based on arterial blood flow within the user's finger. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data. In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for menopause and hot flash detection and treatment based on data collected by a wearable device. In particular, the system 100 illustrated in FIG. 1 may support techniques for detecting hot flashes of a user 102 and/or menopause prediction and causing a user device 106 corresponding to the user 102 to display an indication of the predicted hot flash and/or menopause metric. For example, as shown in FIG. 1, User 1 (user 102-a) may be associated with a wearable device 104-a (e.g., ring 104-a) and a user device 106-a. In this example, the ring 104-a may collect data associated with the user 102-a, including temperature, heart rate, sleep, and the like. In some aspects, data collected by the ring 104-a may be used to estimate a future hot flash during which User 1 experiences a hot flash and/or used to predict menopause onset during which User 1 will experience menopause symptoms. Predicting the menopause onset and/or hot flash may be performed by any of the components of the system 100, including the ring 104-a, the user device 106-a associated with User 1, the one or more servers 110, or any combination thereof. Upon estimating the hot flash and/or menopause metric, the system 100 may selectively cause the GUI of the user device 106-a to display an indication of the estimated future hot flash and/or menopause metric.

In some implementations, upon receiving physiological data (e.g., including temperature data and heart rate data), the system 100 may identify one or more physiological indications of a hot flash experienced by the user based on the physiological data satisfying one or more thresholds. In some examples, the system 100 may determine a metabolic efficiency metric associated with the user based on the received physiological data. In such cases, the system 100 may determine a menopause metric for the user based on the metabolic efficiency metric. The menopause metric may be associated with a relative probability that the user will experience menopausal symptoms. In some cases, the menopause metric may be associated with a relative probability that the user will experience premenopausal, perimenopausal, menopausal, or postmenopausal symptoms. The menopause metric may indicate where the user is in a timeline of experiencing symptoms associated with menopause. For example, the menopause metric may indicate whether the user is in premenopause perimenopause, menopause, or postmenopause.

In some cases, the system 100 may prompt User 1 (e.g., via a GUI of the user device 106) to confirm whether the user 102-a experienced perimenopausal or menopausal symptoms (e.g., hot flashes) or not, and may selectively adjust Readiness Scores for the user 102-a based on confirmation that the user experienced perimenopausal or menopausal symptoms. In some implementations, the system 100 may generate alerts, messages, or recommendations for User 1 (e.g., via the ring 104-a, user device 106-a, or both) based on the predicted perimenopausal or menopausal symptoms, where the alerts may provide insights regarding the predicted perimenopausal or menopausal symptoms, such as a timing and/or duration of the predicted perimenopausal or menopausal symptoms. In some cases, users may provide an input (e.g., a hot flash tag) and the system may associate the input with a physiological outcome and inform users about such associations (e.g., every time a user tags “caffeine.” the user later tags “hot flash”).

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

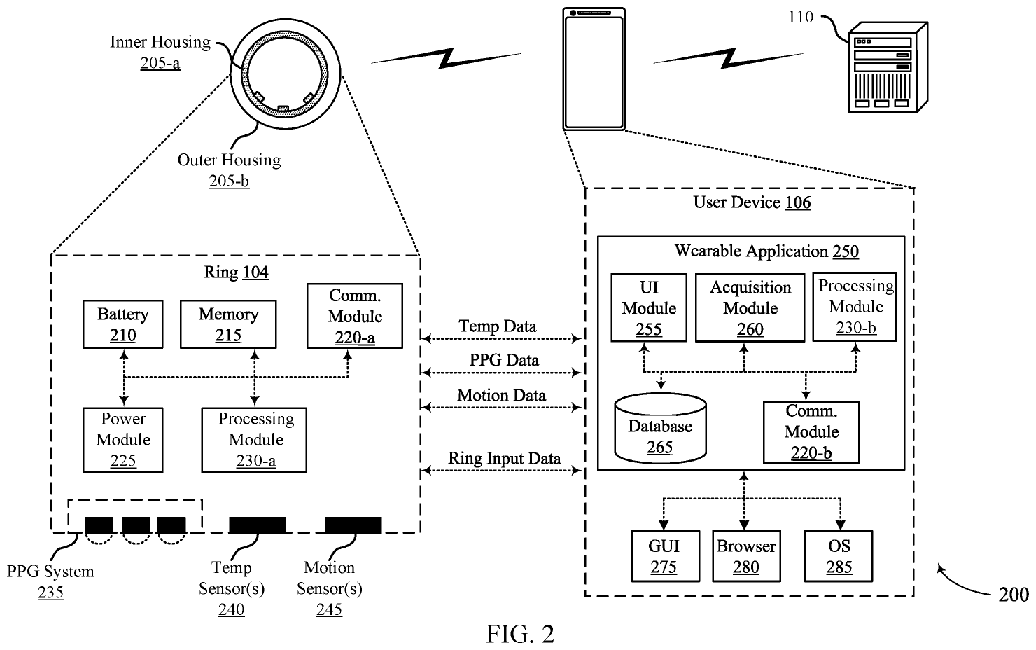

FIG. 2 illustrates an example of a system 200 that supports techniques for menopause and hot flash detection and treatment in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

System 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205, that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, that may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 that the optical receiver(s) receive transmitted light that is reflected through the region of the user's finger. In some implementations, the PPG system 235 may be configured as a transmissive PPG system 235 that the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include LEDs. The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform, that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for predicting perimenopausal or menopausal symptoms onset and offset (e.g., including hot flashes). In particular, the respective components of the system 200 may be used to identify one or more physiological indications of a hot flash experienced by the user based on received physiological data satisfying one or more thresholds. In some examples, the system 200 may determine a metabolic efficiency metric associated with the user based on the received physiological data and determine a menopause metric associated with a relative probability that the user will experience perimenopausal or menopausal symptoms. The menopause metric may indicate where the user is in a timeline of experiencing symptoms associated with menopause. For example, the menopause metric may indicate whether the user is in premenopause perimenopause, menopause, or postmenopause. The perimenopausal or menopausal symptoms (e.g., including a hot flash) for the user may be predicted by leveraging temperature sensors, heart rate sensors, and the like, on the ring 104 of the system 200. In some examples, pre-menopausal symptoms or an end of menstruation may be predicted by leveraging temperature sensors, heart rate sensors, and the like, on the ring 104 of the system 200. In such cases, the system 200 may alert the user that they may be entering menopause based on the end of menstruation. In some examples, menopause may be predicted based on changes in menstrual cycle patterns, temperature, heart rate, HRV, and the like.

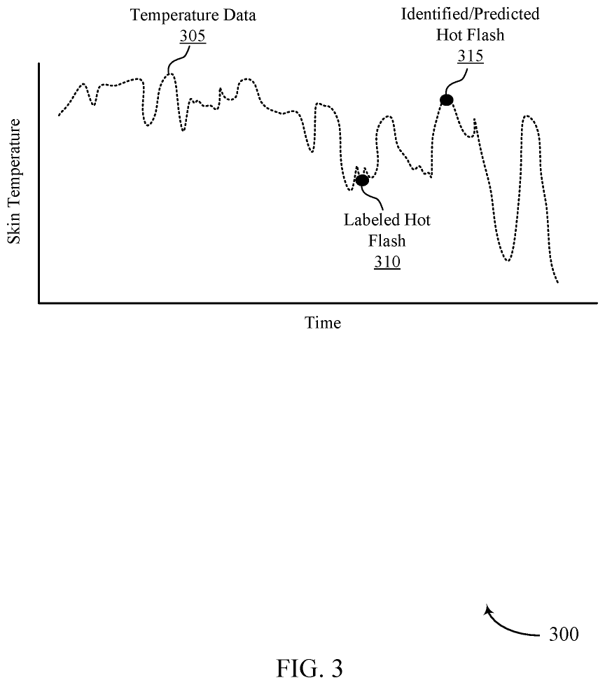

For example, as noted previously herein, the ring 104 of the system 200 may be worn by a user to collect physiological data from the user, including temperature, heart rate, and the like. The ring 104 of the system 200 may collect the physiological data from the user based on arterial blood flow. The physiological data may be collected continuously. In some implementations, the processing module 230-a may sample the user's temperature continuously throughout the day and night. Sampling at a sufficient rate (e.g., one sample per minute) throughout the day may provide sufficient temperature data for analysis described herein. In some implementations, the ring 104 may continuously acquire temperature data and heart rate data (e.g., at a sampling rate). Data collected by the ring 104 may be used to determine when the user experiences an onset of perimenopausal or menopausal symptoms. Predicted hot flashes may be further shown and described with reference to FIG. 3.

FIG. 3 illustrates an example of a timing diagram 300 that supports techniques for menopause and hot flash detection and treatment in accordance with aspects of the present disclosure. The timing diagram 300 may implement, or be implemented by, aspects of the system 100, system 200, or both. For example, in some implementations, the timing diagram 300 indicating a relative timing of a predicted hot flash may be displayed to a user via the GUI 275 of the user device 106, as shown in FIG. 2.

In some cases, information acquired and determined by the system 200 (e.g., wearable devices and applications) may be used to identify relevant events for users experiencing menopause and perimenopause. Example information may include, but is not limited to, user-provided information (e.g., tags in an application) and sensor acquired information. Sensor acquired information may include skin temperature, HR, HRV, electrodermal activity (EDA), galvanic skin response, SpO2, actigraphy (sleep and activity), and/or geolocation. In some implementations, multiple sensors may be combined to correct artifacts and triangulate different aspects of physiologic functions. In such cases, machine learning approaches may use raw signals or featured data for biological signals like skin temperature data 305, heart rate, respiratory rate, electrodermal activity, galvanic skin response, or a combination thereof, to detect hot flashes

In some cases, example features associated with relevant reproductive aging events may include, but are not limited to hot flash detection, hot flash detection and/or prediction, associating and surfacing the relationship between physiological patterns (e.g., sleep quality, exercise) and behavioral patterns (e.g., coffee consumption, smoking) and perimenopausal or menopausal symptoms, alerting users that they are entering a perimenopause or menopause phase, alerting users when they might be experiencing increase in symptoms and/or what actions they may take to prevent/reduce symptoms (e.g., exercise, HRT), or a combination thereof.

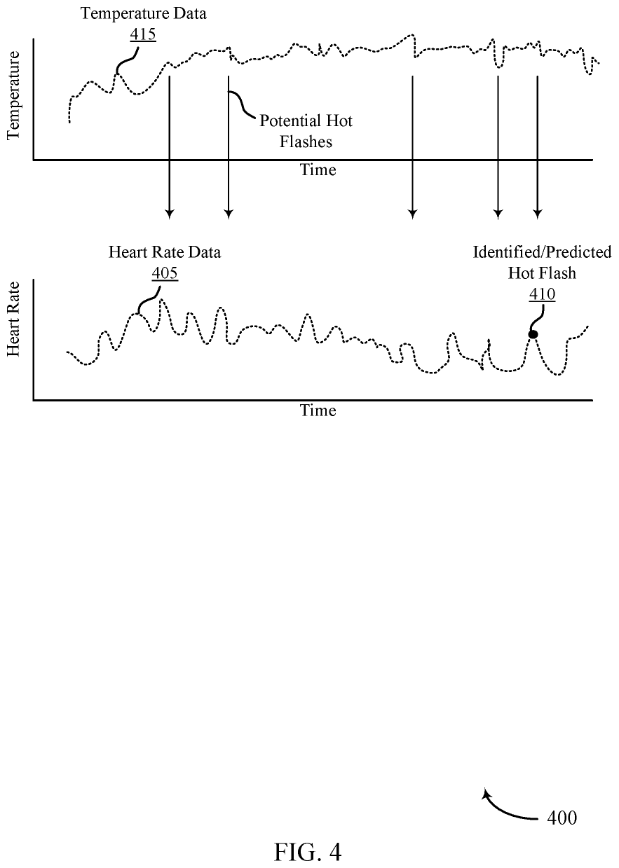

For example, the system 200 may identify one or more physiological indications of a hot flash experienced by the user based on physiological data satisfying one or more thresholds. In some implementations, the physiological indications of a hot flash may be detected by continuous skin temperature data 305. The skin temperature data 305 may be collected at 1-minute increments (e.g., frequency). The hot flashes may be evident in the temperature data 305 during the night when the skin temperature data 305 is stable (e.g., maintained at a temperature). In some cases, HR, activity information, and respiratory rate may be used for detecting hot flashes, as described with reference to FIG. 4.

In some cases, an increase in temperature (e.g., temperature data 305) may indicate an onset of menopause. In such cases, an increase in temperature data 305 associated with the onset of menopause may reduce erroneous illness detection. For example, the system 200 may detect an increase in temperature data 305 and determine that the user is entering menopause rather than detecting an onset of an illness.

As will be described in further detail herein, the system 200 may be configured to identify and/or predict hot flashes. As such, the timing diagram 300 illustrates a relationship between a user's temperature data 305 and an identified and/or predicted hot flash 315. In this regard, the “dots” illustrated in the timing diagram 300 may be understood to refer to a user's measured skin temperature (e.g., absolute temperature data). In this regard, the dots illustrated in the timing diagram 300 may be referred to as “temperature data 305.” In some cases, the system 200 may determine, or estimate, temperature data 305 for a user based on temperature data 305 for the user collected via the ring 104.

The timing diagram 300 shown in FIG. 3 illustrates a relative timing of identified/predicted hot flashes 315 relative to a traditional calendar day. In particular, the timing diagram 300 illustrates identified/predicted hot flashes 315 (e.g., a day during which the user experiences a hot flash) for a user throughout one calendar day. A predicted hot flash 315 may be defined as a duration of time during which the user is predicted to experience a hot flash. For example, a user may experience a hot flash 320 during a perimenopause phase or menopause phase. The timing diagram 300 may include at least three predicted hot flashes 315 as described below in further detail. The timing diagram 300 may also include at least one labeled hot flash 310, as described below in further detail.

In some cases, the system 200 (e.g., ring 104, user device 106, server 110) may receive physiological data associated with the user from a wearable device 104. The physiological data may include at least temperature data 305 and also may include heart rate data along with other physiological measurements or derived values. The temperature data 305 may be continuously collected by the wearable device. The physiological measurements may be taken continuously throughout the day and/or night. For example, in some implementations, the ring 104 may be configured to acquire physiological data (e.g., temperature data 305, heart rate, and the like) continuously in accordance with one or more measurement periodicities throughout the entirety of each day/sleep day. In other words, the ring 104 may continuously acquire physiological data from the user without regard to “trigger conditions” for performing such measurements. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body or if the user were manually taking their temperature once per day.

The system 200 may receive additional physiological data associated with the user from the wearable device. In such cases, the system 200 may determine baseline physiological data for the user based on the additional physiological data. The one or more thresholds may be determined or calculated based on the baseline physiological data for the user. In some examples, the system 200 may identify a baseline temperature associated with the user based on receiving the physiological data. The baseline temperature may include a nighttime temperature baseline. In other examples, the system 200 may identify a nighttime temperature baseline for a group of users, and identify the baseline temperature associated with the user based on identifying the nighttime temperature baseline. In such cases, the system may determine a nighttime baseline temperature based on average nighttime baseline temperatures of a group of people. The baseline temperature may be, for example, 35-36.5° C., that may be within a range of the actual core body temperature of the user.

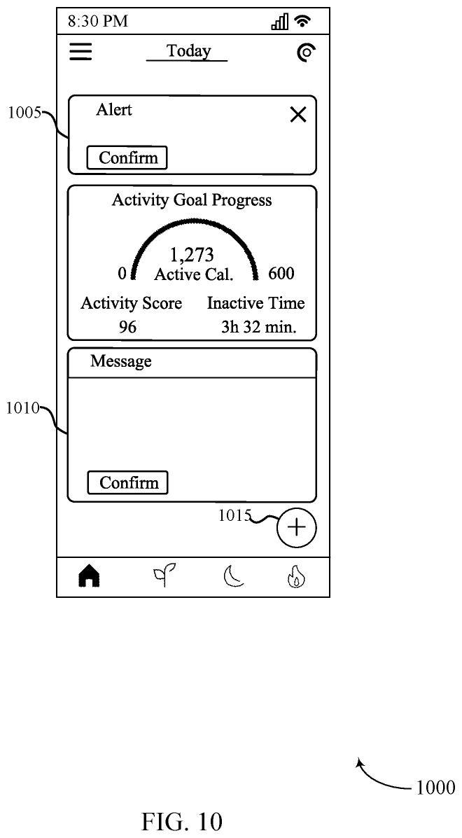

In some cases, the system 200 may identify hot flash triggers based on previously-identified hot flashes and supplemental data received from the user (e.g., user experiences more hot flashes after they've consumed alcohol or consumed spicy food). The system 200 may determine a hot flash risk metric for the user based on the additional physiological data. The hot flash risk metric may be associated with a relative probability that the user will experience a potential hot flash. For example, the user may receive an alert that may indicate a date of when the hot flash is predicted, a date range including when the hot flash is predicted, a duration of time until the hot flash, and the like. In such cases, the system 200 may display an indication of the potential hot flash based at least in part on the hot flash risk metric satisfying hot flash prediction threshold, as described with reference to FIG. 10.

In some cases, a hot flash may be detected with wavelet adapting. In other examples, a hot flash waveform may be matched to a skin temperature signal (e.g., temperature data 305) to filter out hot flash related variations in the received signal. In some implementations, the system 200 may utilize classifiers (e.g., machine learning classifiers) to identify the predicted hot flash 315. In particular, the system 200 may train a classifier to identify the predicted hot flash 315 for a user based on inputted physiological data for the user. For example, in some cases, physiological data collected from a user (e.g., including the temperature data 305 and/or heart rate data) may be inputted into a classifier (e.g., machine learning classifier), where identifying the predicted hot flash 315 is based on inputting the physiological data into the machine learning classifier (e.g., the classifier is configured to identify the predicted hot flash 315).

A hot flash may be detected based on pattern recognition. For example, the skin temperature (e.g., temperature data 305) of the user may suddenly and significantly rise (e.g., above the user's typical or baseline sleep temperature). In some implementations, the system 200 may identify and/or predict a hot flash in cases where the user's temperature exhibits a sudden rise (e.g., temperature increase above a user's baseline temperature range), followed by a dampening of the temperature back to the user's baseline temperature/temperature range. In such cases, the sudden temperature increase and subsequent slower temperature decrease may take place over a relatively short amount of time, and may vary from user to user. As such, the system 200 may be configured to learn and identify patterns in acquired physiological data for each respective user which are indicative of hot flashes for the respective user.

If the user experiences a hot flash during sleep that may be indicated by the user experiencing night sweats, the sleep may be disturbed. The duration of the hot flash may last for a minute or up to five minutes. In such cases, the length of the hot flash may be illustrated by spikes in the temperature signal, and for some users, the hot flashes may occur multiple times within a short period of time. If the heart rate is measured simultaneously with the temperature, the heart rate may be expected to rise by about twenty percent. The system 200 may be configured to identify patterns between temperature data and heart rate data to identify and/or predict hot flashes 315.

In some implementations, user inputs received from a user (e.g., via GUI 275 of the user device 106) may be used to further train a classifier to identify the predicted hot flash 315. In other words, a user may be able to generate user inputs that may then be used to train the machine learning classifier. For example, the classifier may identify/predict the hot flash 315 for a user based on received physiological data. Subsequently, the system 200 may prompt the user (e.g., via the GUI 275) to confirm or deny whether the user experienced the predicted hot flash 315, and the user inputs (e.g., confirmation or denial of the predicted hot flash 315) may be used to further train the classifier to become more effective at accurately identify the predicted hot flash 315.

The system 200 may be configured to detect multiple predicted hot flashes 315 within a calendar day, a calendar month, or both. Moreover, the system 200 may be configured to adjust scores for the user (e.g., Sleep Scores, Readiness Scores) based on the predicted hot flash 315 detected throughout the day. In addition to supporting a diverse group of users, being able to detect predicted hot flashes 315 may provide more accurate health information to the users, and may improve business-to-business (B2B) use cases, such as illness detection and fertility initiatives. Moreover, as noted previously herein, the detection of a “predicted hot flash” for a user may enable the system 200 to determine a more complete and accurate picture of the user's overall health (e.g., more accurate Readiness Scores). As such, by enabling more complete and accurate Readiness Scores, techniques described herein may enable the system 200 to provide improved insights and guidance to the user that better correlate to the user's overall health.

For the purposes of the present disclosure, the terms “hot flash,” “menopause metric,” “menopausal symptoms,” “perimenopausal symptoms,” and like terms, may be used interchangeably. In some cases, the system 200 (e.g., user device 106, server 110) may be configured to receive data collected from a user via the ring 104, and predict an onset of perimenopausal or menopausal symptoms.

Referring to the system 200 illustrated in FIG. 2, the ring 104 may be worn by a user and may collect data associated with the user throughout a day. The ring 104 may collect data (e.g., temperature, heart rate) and transmit collected data to the user device 106. In some cases, the user device 106 may forward (e.g., relay, transmit) the data received from the ring 104 to the servers for processing. Additionally, or alternatively, the user device 106 and/or the ring 104 may perform processing on the collected data.

Continuing with the same example, the ring 104, the user device 106, the servers 110, or any combination thereof, may estimate a future predicted hot flash 315 based on the collected data. Upon estimating the predicted hot flash 315, the servers 110 may transmit an indication of the predicted hot flash 315 to the user device 106. Alternatively, in cases where the user device 106 performs data processing, the user device 106 may generate the indication of the predicted hot flash 315. In this example, the next time the user opens the wearable application 250, an indication of the predicted hot flash 315 may be presented to the user via the GUI 275 of the user device 106. This may be further understood with reference to FIG. 10.

FIG. 4 illustrates an example of a timing diagram 400 that supports techniques for menopause and hot flash detection and treatment in accordance with aspects of the present disclosure. The timing diagram 400 may implement, or be implemented by, aspects of the system 100, system 200, or both. For example, in some implementations, the timing diagram 400 indicating an identified and/or predicted hot flash 410 may be displayed to a user via the GUI 275 of the user device 106, as shown in FIG. 2.

In some implementations, the system 200 may use other signals and physiological parameters combined with temperature. For example, the system 200 may combine information from a variety of other sensors and signals to improve the detection or prediction of identified/predicted hot flashes 410. In some examples, the system 200 may include features or frequency extractions from a PPG signal or actigraphy sensors and activity detection, among others. In some implementations, the system 200 may use HRV or heart rate data 405 to identify the identified/predicted hot flash 410.

In some cases, the system 200 may identify a first change in the temperature data within a first time interval is greater than a temperature change threshold. The system 200 may identify a second change in the heart rate data 405 within the first time interval is greater than a heart rate change threshold. In such cases, the system 200 may identify hot flashes based on dramatic changes in temperature (e.g., temperature drops) that overlap with a dramatic changes in heart rate data 405 (e.g., heart rate increases and/or includes a quickening pulse).

For example, various heart rate data 405 and HRV features may be derived from a PPG, that may be useful in cycle detection. Similarly, as described with reference to FIG. 3, the system 200 may acquire and analyze temperature data 415 for the user. In particular, the timing diagram 400 illustrates how trends or correlations between temperature data 415 and heart rate data 405 may be used to identify or predict hot flashes.

The system 200 may extract a variety of HRV frequency bands and features, such as the root mean squared difference between successive intervals (RMSSD), the high frequency (HF), low frequency (LF), very low frequency (VLF), and respiration rate frequencies (e.g., breathing rate). The system 200 may extract peak power in a given frequency range measured over appropriate intervals during the night (e.g., approximately 1 minute for HF, approximately 5 minutes for LF, etc.). The system 200 may use the frequency-based features to strengthen a cycle phase detection or prediction algorithm.

As such, the timing diagram 400 illustrates a relationship between a user's heart rate data 405 and identified/predicted hot flash 410. In this regard, the “curved line” illustrated in the timing diagram 400 may be understood to refer to a user's measured heart rate. In this regard, the curved line illustrated in the timing diagram 400 may be referred to as “heart rate data 405.” In some cases, the system 200 may determine, or estimate, heart rate data 405 for a user based on heart rate data 405 for the user collected via the ring 104.

As shown in FIG. 4, the system 200 may be configured to identify or predict hot flashes by identifying trends, relationships, or other features between the user's temperature data 415 and heart rate data 405. For example, as shown in FIG. 4, periods of increasing skin temperature followed by sharp decreases of skin temperature may indicate potential hot flashes. Moreover, the system 200 may be configured to identify an increased likelihood of a hot flash if such periods of increasing/decreasing skin temperature are found to occur at (approximately) the same time as increases in the user's heart rate data 405.

The system 200 may implement a variety of models and algorithms to implement a method for detecting hot flashes. For example, the system 200 may implement neural networks, image analysis, prospective prediction algorithms, and/or subgroup detection and clustering for precision medicine/personalized insights. In some implementations, the system 200 may use alternative algorithms to predict and/or detect the timing of hot flashes. For example, the system 200 may take the heatmaps and feed the heatmaps into a convolutional neural network (CNN), auto-encoder, or various deep learning approaches appropriate for image analysis. The system 200 may feed the subsequent features into a time series detection model. In some implementations, the system 200 may use various temperature features, such as the temperature distributions, their ultradian rhythms, or their timing relative to bedtime, sleep staging, or phase of sleep (e.g., early, late, etc.) to predict a woman's likelihood to experience a hot flash.

In some implementations, the system 200 may use various temperature features to cluster women into subgroups. For example, the system 200 may apply various machine learning clustering algorithms, such as factor analysis, principal components, K-means, K-nearest neighbors, or agglomerative clustering or more to assign women to a subgroup. These subgroups may provide the basis for personalized insights in the wearable application. For example, the system 200 may be able to detect women with various reproductive-related conditions like ovarian aging.

In some implementations, one or more devices may predict treatment success (e.g., hormone replacement treatment or exercise for treatment of hot flashes). For example, a device (e.g., application) may operate in a reproductive aging mode and/or healthy aging mode in the context of users seeking treatment to relieve symptoms related to reproductive aging (e.g., brain fog, sleep disturbance, hot flashes, and the like). Clinical reproductive health, menopause, andropause, and wellbeing centers may quantify various fertility-relevant outcomes and reproductive aging-relevant outcomes including menstrual cycles, hormone levels, sleep, brain fog, and hot flashes. Data may be converted into a set of physiologic machine learning features that may be incorporated into a treatment (e.g., HRT, exercise) success prediction algorithm that may predict likelihood of reducing symptoms. In some cases, example features may include the average cycle length (e.g., based on temperature-estimated period start dates), minimum and maximum cycle lengths, number of anovulatory cycles detected, cycle irregularity (e.g., standard deviation of the cycle length distribution), basic demographics, body mass index, and age-adjusted versions of these features. In some examples, physiologic-based features may be used in combination with additional information regarding sleep, activity, metabolic, or cardiovascular parameters, as well as in combination with clinically gathered bloodwork values. In some implementations, provider identifiers assigned to the treatment location or treating physician, featurization of the treatment protocol, and biological/clinical read-outs may be incorporated for additional predictive value of predicted hot flashes 410.

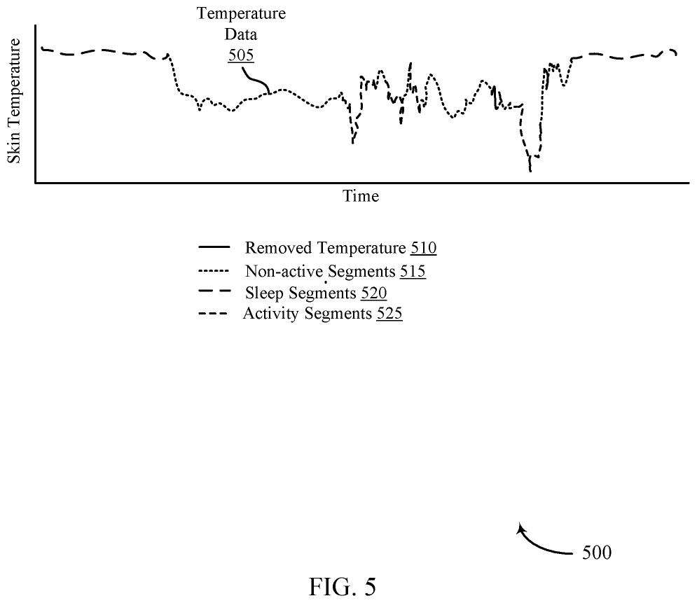

FIG. 5 illustrates an example of a timing diagram 500 that supports techniques for menopause and hot flash detection and treatment in accordance with aspects of the present disclosure. The timing diagram 500 may implement, or be implemented by, aspects of the system 100, system 200, or both. For example, in some implementations, the timing diagram 500 indicating the effects of activity on skin temperature and MET may be displayed to a user via the GUI 275 of the user device 106, as shown in FIG. 2.

In some cases, activity such as high intensity interval training (HIIT) may be a behavioral treatment for perimenopausal hot flashes and other symptoms. HIIT may improve metabolic efficiency by improving the function of mitochondria, the cellular organelles that produce cellular energy to fuel activity. During the menopausal transition, the withdrawal of estrogen may have an adverse effect on glucose regulation, thereby reducing metabolic efficiency. In such cases, individuals experiencing the menopausal transition may gain weight around the mid-section (e.g., abdominal fat). The decrease in estrogen and metabolic efficiency may also lead to symptoms of fatigue, hot flashes, sleep disruption, and resulting mood or cognitive issues. For example, perimenopausal or menopausal symptoms (e.g., hot flash severity or frequency) may correlate with reductions in age- or exercise-associated metabolic efficiency. In such cases, wearable-derived metrics of metabolic efficiency may be the reactivity and/or recovery of metabolic equivalents (METs) and temperature (e.g., temperature data 505).

Techniques to quantify and measures the effects of thermoregulatory narrowing in the context of exercise may be desired. In some cases, per unit increase in METs in response to activity may result in the temperature (e.g., temperature data 505) dropping more than expected, thereby reflecting an increased thermoregulatory reactivity to an exercise-induced increase in core body temperature. The system 200 may determine that greater negative slopes of decrease in temperature data 505 during activity (e.g., thermal reactivity to activity, exercise-induced thermogenesis, and the like) may be associated with greater perimenopausal or menopausal symptoms, such as more frequent or severe hot flashes, sleep disruption, mood symptoms, and the like. In some implementations, a decrease in temperature data 505 during activity may be normalized to the type of activity or the concurrent increase in METs.

In some cases, when activity or METs increase, the temperature data 505 drops. The system 200 may detect a drop in skin temperature (e.g., temperature data 505) during activity segments 525. In such cases, the user may experience an opening of the blood vessels to release the increased core temperature heat generated by exercise. During sleep segments 520, the temperature data 505 may be elevated (e.g., at higher temperatures) as compared to during the activity segments 525. In other examples, the temperature data 505 may be elevated (e.g., at higher temperatures) as compared to during the activity segments. In such cases, when activity or METs decrease, the temperature data 505 increases. In some cases, the system 200 may remove temperature 510.

In some examples, the sleep segments 520 may indicate whether the user will experience perimenopausal or menopausal symptoms. For example, the system 200 may receive the physiological data associated with the user from the wearable device where the physiological data includes sleep data. In some cases, the system 200 may determine a baseline sleep threshold for the user (e.g., baseline sleep data). The system 200 may determine that the sleep data deviates from the baseline sleep threshold for the user. In such cases, the system 200 may determine the menopause metric in response to determining that the sleep data deviates from the baseline sleep threshold for the user.

In such cases, the system 200 may determine the menopause metric based on determining that the received sleep data is less than or greater than the baseline sleep threshold for the user. For example, the system 200 may determine that the sleep architecture from the received sleep data deviates from a baseline sleep architecture threshold for the user. The system 200 may determine the menopause metric in response to determining that the sleep architecture from the received sleep data from a baseline sleep architecture threshold for the user.

The sleep architecture may include an amount (or proportion) of REM sleep, an amount of deep sleep, an amount of total sleep, restfulness, sleep latency, sleep density, an amount of wake periods, or a combination thereof. For example, the sleep architecture may include a short REM latency (e.g., the time it takes to enter REM sleep from sleep onset), difficulties initiating sleep, decreased sleep continuity (i.e., increased number of awakenings), decreased slow-wave sleep (i.e., decreased percentage of stage 3 or 4 sleep), or a combination thereof.

For example, the system 200 may determine that the amount of deep sleep, amount of REM sleep, total sleep time, restfulness, sleep latency, sleep density, amount of wake periods, or a combination thereof deviates from a sleep baseline for the user. In some examples, the system 200 may determine the menopause metric based on determining that the amount of deep sleep, amount of REM sleep, total sleep time, restfulness, sleep latency, sleep density, an amount of wake periods, or a combination thereof deviates from the sleep baseline for the user.

The sleep baselines (e.g., baseline sleep threshold, baseline sleep architecture thresholds, and the like) may be tailored specific to the user based on historical data acquired by the system 200. For example, these baselines may represent baseline or average values of physiological parameters or typical trends of physiological values throughout a user's premenopausal, perimenopausal, menopausal, or postmenopausal periods, that may differ from the user's normal or non-menopausal baselines. In some cases, the baselines may differ throughout the user's menopausal period (e.g., based on the different stages of menopause) for each physiological parameter. In some cases, the baselines may be based on known standards, averages among users, and/or demographic-specific.

In some implementations, the system 200 may determine a metabolic efficiency metric associated with the user based on the received physiological data. The received physiological data may be an example of temperature data 505, HR data, HRV data, and the like. In some cases, the system may determine a menopause metric for the user based on the metabolic efficiency metric. In some examples, the metabolic efficiency metric may be indicative of a relative efficiency that cells of the user produce cellular energy. In some cases, the metabolic efficiency metric may be associated with a thermoregulatory ability of the user to regulate internal body temperature. Moreover, the menopause metric may be associated with a relative probability that the user will experience menopausal symptoms. In some cases, the menopause metric may be associated with a relative probability that the user will experience premenopausal, perimenopausal, menopausal, or postmenopausal symptoms. The menopause metric may indicate where the user is in a timeline of experiencing symptoms associated with menopause. For example, the menopause metric may indicate whether the user is in premenopause, perimenopause, menopause, or postmenopause. The system 200 may cause a GUI of a user device to display one or more messages associated with the menopause metric. This may be further understood with reference to FIG. 10.

The system 200 may determine a change in the metabolic efficiency metric associated with the user based on identifying the metabolic efficiency metric. In such cases, determining the menopause metric may be based on the change in the metabolic efficiency metric. For example, the system 200 may identify a menopause risk metric based on changes in the user's metabolic efficiency (e.g., if marathon runner starts showing decreased metabolic efficiency, the decrease in the user's metabolic efficiency may be more indicative of menopause).

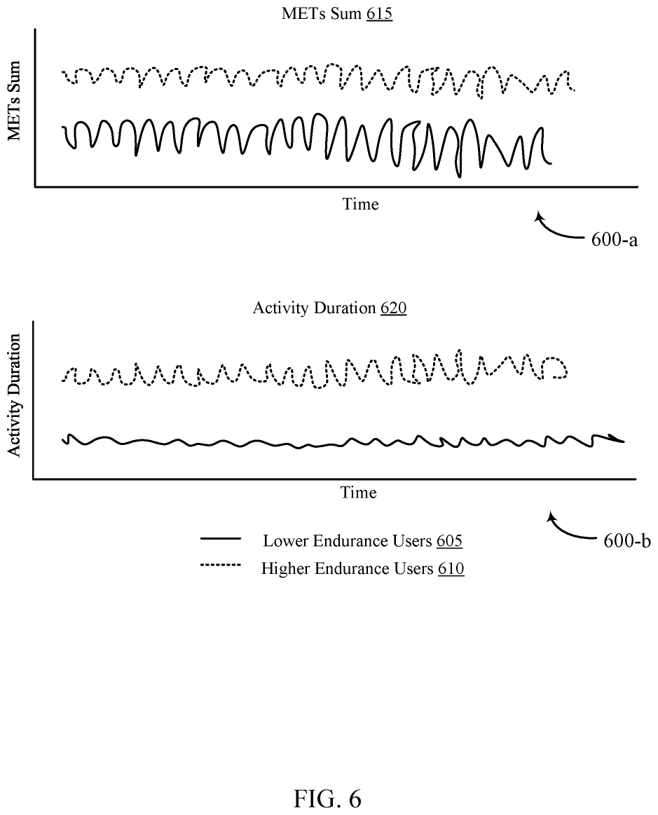

FIG. 6 illustrates an example of timing diagrams 600 that support techniques for menopause and hot flash detection and treatment in accordance with aspects of the present disclosure. The timing diagrams 600 may implement, or be implemented by, aspects of the system 100, system 200, or both.

The timing diagram 600-a may include the METs sum 615 over a period of time. The timing diagram 600-b may include an activity duration 620 over a period of time. In some cases, a group of users may be divided into two groups including lower endurance (e.g., lower activity) users 605 and higher endurance (e.g., higher activity) users 610. The user's METs sum 615 may be quantified over a 6-month period of time (e.g., January 1st through mid-July 2020). The users in the top 50th percentile for exercise duration over that period of time may be identified as higher endurance users 610. The users in the lower 50th percentile may be identified as lower endurance users 605. The system 200 may determine how duration of exercise correlates to total METs sum 615 and the duration of activity over time. The total METs sum 615 may be an example of a total energy expenditure detected by the accelerometer (e.g., of the ring 104). For example, individuals who walk regularly may have longer duration but expend less METs than individuals who attend athletic classes (e.g., spin classes, HIIT classes, and the like).

In some cases, the system 200 may determine that endurance exercise may result in increased metabolic efficiency. For example, higher endurance users 610 may expend about 200 more METs on a daily basis than lower endurance users 605. Higher endurance users 610 may also exercise for longer durations. In such cases, the system 200 may determine that the endurance exercise of the higher endurance users 610 may be consistent over the 6-month period on average. In some cases, the system 200 may determine that there is a weekly rhythm to how long and hard individuals exercise, thereby indicating a weekday-weekend seasonality over the period of time.

The system 200 may determine the users' average activity duration during a period of time (e.g., January 1st through Jul. 14, 2020) and divide the group of users into the top and bottom 50 percentiles by users with high duration across the period of time (e.g., higher endurance users 610) and users with low duration across the period of time (e.g., lower endurance users 605). In some cases, the system 200 may determine whether fitness (e.g., exercise duration) may be associated with more efficient acute metabolic efficiency during the first 20 minutes of exercise (e.g., reactivity). In such cases, the system 200 may select the most active period of the user's day based on the longest period of METs sum 615 on a given day.

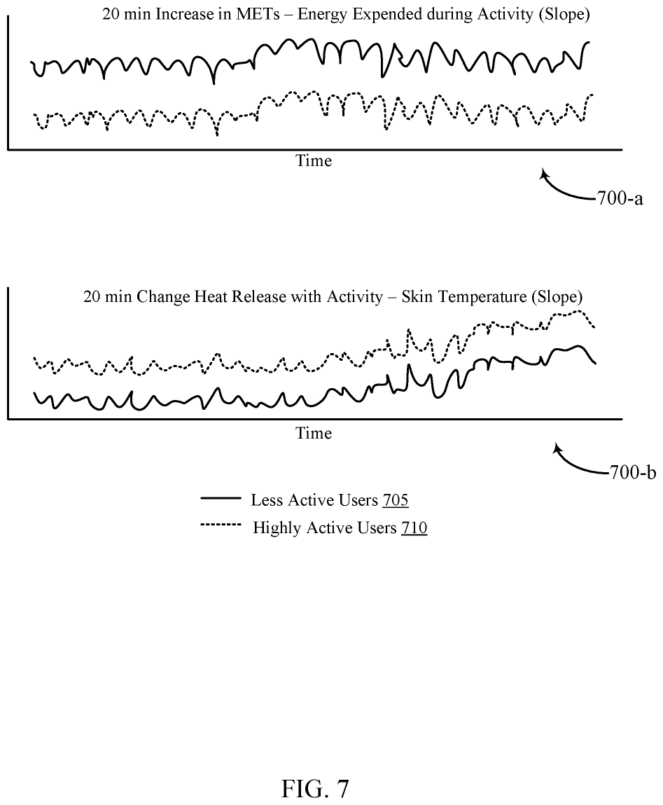

FIG. 7 illustrates an example of timing diagrams 700 that support techniques for menopause and hot flash detection and treatment in accordance with aspects of the present disclosure. The timing diagrams 700 may implement, or be implemented by, aspects of the system 100, system 200, or both.

In some cases, a group of users may be divided into two groups including less active users 705 and highly active users 710. In some cases, the system 200 may determine that endurance exercise results in increased metabolic efficiency. For example, for a user that exhibits lower metabolic efficiency based on timing diagram 600, an increase in activity may result in a greater slope of increase in METs and a greater slope of decrease in temperature. In such cases, the body may have to work harder metabolically to generate the energy to sustain the activity. For example, the system 200 may determine that the result is a greater increase in METs and a greater decrease in skin temperature.

Users that exercise for longer periods of time (e.g., highly active users 710) may develop more efficient skeletal muscle and/or mitochondrial efficiency (e.g., higher metabolic efficiency). In such cases, there is a reduced energy cost for the same amount of physical activity. Users that are less active (e.g., less active users 705) may exercise for shorter durations of time and may have to work harder to generate energy when they are exercising (e.g., experiencing higher increases in METs). In such cases, less active users 705 may generate more heat to release and see greater skin temperature reductions to compensate. The system 200 may help user's understand and tune the user's fitness by using METs and temperature to estimate metabolic efficiency during exercise.

In some implementations, the system 200 may identify a time interval during which the user is engaged in physical activity based on the physiological data. The system 200 may determine a first rate of change associated with MET data for the user during the time interval based on the physiological data. In some cases, the system 200 may determine a second rate of change associated with temperature data for the user during the time interval based on the physiological data. In such cases, determining the metabolic efficiency metric may be based on the first rate of change, the second rate of change, or both. For example, the system 200 may determine metabolic efficiency based on slopes of MET and temperate change during periods of activity.

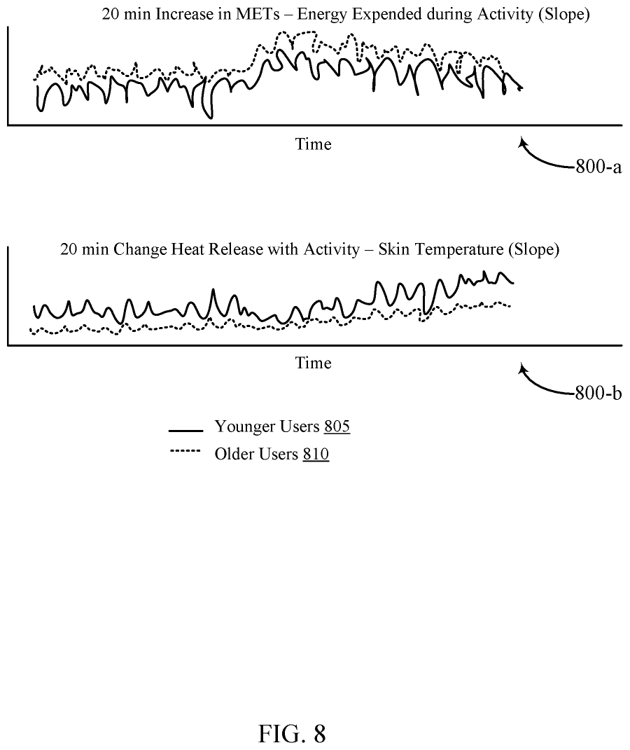

FIG. 8 illustrates an example of timing diagrams 800 that support techniques for menopause and hot flash detection and treatment in accordance with aspects of the present disclosure. The timing diagrams 800 may implement, or be implemented by, aspects of the system 100, system 200, or both.

In some cases, a group of users may be divided into two groups including younger users 805 and older users 810. In some cases, the system 200 may determine that the age of the individual reduces metabolic efficiency. Women enter the menopausal transition as a function of age (e.g., around age 50 or 51). Perimenopause may start earlier (e.g., in the 30's or the 40's). In such cases, the system 200 may determine an age-associated decrease in metabolic efficiency. For example, the timing diagram 800-a may illustrate that older users 810 expend more METs than younger users 805. The timing diagram 800-b may illustrate that older users 810 may have greater skim temperature reduction during activity than younger users 805. In such cases, the system 200 may determine that the older users 810 may be working harder metabolically to support the activity (e.g., older users 810 may exhibit decreased metabolic efficiency as compared to younger users 805).

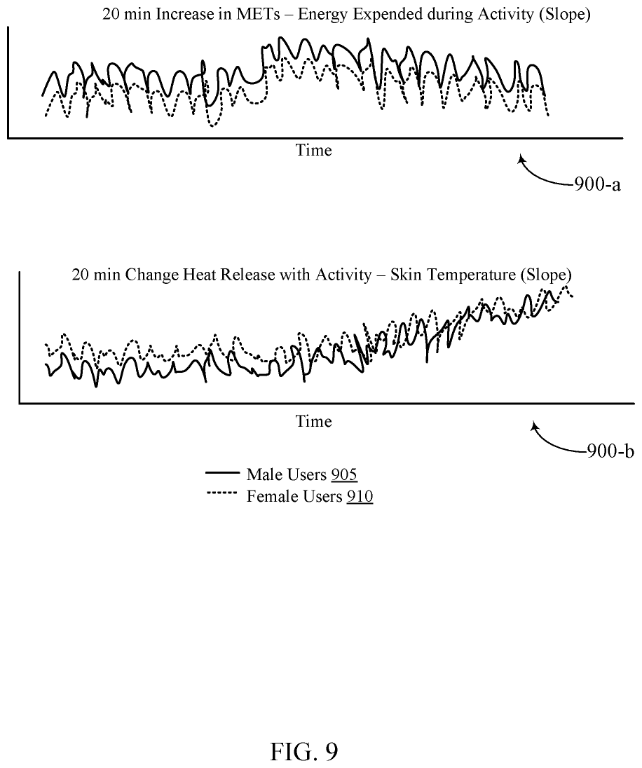

FIG. 9 illustrates an example of timing diagrams 900 that support techniques for menopause and hot flash detection and treatment in accordance with aspects of the present disclosure. The timing diagrams 900 may implement, or be implemented by, aspects of the system 100, system 200, or both.

In some cases, a group of users may be divided into two groups including male users 905 and female users 910. The timing diagram 900-a may illustrate that the male users 905 may have a greater increase in METs than the female users 910. The increase may be based on that male users 905 may be larger weight or mass than female users 910 and may generate more energy to move a larger body. Timing diagram 900-b may illustrate similar changes in skin temperature during activity (e.g., reactivity) for male users 905 and female users 910.

FIG. 10 illustrates an example of a GUI 1000 that supports techniques for menopause and hot flash detection and treatment in accordance with aspects of the present disclosure. The GUI 1000 may implement, or be implemented by, aspects of the system 100, system 200, timing diagrams 300, 400, 500, 600, 700, 800, 900, or any combination thereof. For example, the GUI 1000 may be an example of a GUI 275 of a user device 106 (e.g., user device 106-a, 106-b, 106-c) corresponding to a user 102.

In some examples, the GUI 1000 illustrates a series of application pages that may be displayed to a user 102 via the GUI 1000 (e.g., GUI 275 illustrated in FIG. 2). The server 110 of system 200 may cause the GUI 1000 of the user device 106 (e.g., mobile device) to display inquiries of whether the user 102 activates the hot flash mode and wants to track their hot flashes experienced. In such cases, the system 200 may generate a personalized tracking experience on the GUI 275 of the user device 106 to estimate the occurrence of a predicted hot flash based on the contextual tags and user questions. Menopausal or perimenopausal symptoms may include, but are not limited to, hot flashes.

Continuing with the examples above, prior to estimating a future hot flash, the user 102 may be presented with the application page upon opening the wearable application 250. The application page may display a request to activate the hot flash mode and enable the system 200 to track the user's hot flashes. In such cases, the application page may display an invitation card where the users are invited to enroll in the hot flash tracking applications. The application page may display a prompt to the user 102 to verify whether the hot flashes may be tracked or dismiss the message 1010 if the hot flashes are not tracked.

The user 102 may be presented with an application page upon selecting “yes” to tracking the hot flashes. The application page may display a prompt to the user 102 to verify the main reason to track the hot flashes (e.g., perimenopause, menopause, etc.). In such cases, the application page may prompt the user 102 to confirm the intent of tracking the hot flashes. For example, the system 200 may receive, via the user device 106, a confirmation of the intended use of the hot flash prediction system.

In some implementations, the application page may display an indication that a hot flash was predicted via alert 1005. In such cases, the application page may include the alert 1005 on the home page. In cases where a user's hot flash day may be predicted, as described herein, the server 110 may transmit an alert 1005 to the user 102, where the alert 1005 is associated with the predicted hot flash for the user 102.

For example, the user 102 may receive an alert 1005, that may indicate a date of when the hot flash is predicted, a date range including the hot flash is predicted, a duration of time until the hot flash, and the like. For example, the alert 1005 may indicate that the hot flash is likely to occur tomorrow, in 5-7 days, or indicate a date of the predicted hot flash (e.g., predicted hot flash is August 28th) or a range of dates of the predicted hot flash (e.g., predicted hot flash date April 27-29th). In such cases, the range may include the day of the predicted hot flash and the day before and after the predicted hot flash. The alerts 1005 may be configurable/customizable, such that the user 102 may receive different alerts 1005 based on the predicted hot flash.

In some cases, the user may take remedial action to address the hot flash prior to the system 200 displaying the alert 1005. In such cases, the system 200 may receive physiological data associated with the remedial action, and the system 200 may refrain from displaying the alert 1005 (e.g., override the alert 1005). In some examples, the system 200 may adjust the alert 1005 based on receiving the physiological data associated with the remedial action.

As shown in FIG. 10, the application page may display an indication of whether the predicted hot flash occurred via message 1010. The user 102 may receive message 1010, that may prompt the user 102 to verify whether the hot flash has occurred or dismiss the message 1010 if the predicted hot flash has not occurred. In such cases, the application page may prompt the user 102 to confirm or dismiss the predicted hot flash (e.g., confirm/deny whether the system 200 correctly determined that the user 102 experienced hot flash). For example, the system 200 may receive, via the user device 106 and in response to estimating the future hot flash, a confirmation of the estimated future hot flash. Additionally, in some implementations, the application page may display one or more scores (e.g., Sleep Score, Readiness Score, activity goal progress) for the user 102 for the respective day.

The application pages may display a hot flash card such as a “predicted hot flash confirmation card” that indicates that the predicted hot flash has been recorded. In some implementations, upon confirming that the predicted hot flash is valid, the hot flash may be recorded/logged in an activity log for the user 102 for the respective calendar day. Moreover, in some cases, the hot flash may be used to update (e.g., modify) one or more scores associated with the user 102 (e.g., Sleep Score, Readiness Score). That is, data associated with the predicted hot flash may be used to update the scores for the user 102 for the following calendar day after the hot flash was confirmed. In some cases, the Readiness Score may be updated based on the predicted hot flash.

In some cases, the messages 1010 displayed to the user 102 via the GUI 1000 of the user device 106 may indicate how the predicted hot flash affected the overall scores (e.g., overall Readiness Score) and/or the individual contributing factors. For example, a message 1010 may indicate “It looks like your body is under strain right now, but if you're feeling ok, doing a light or medium intensity exercise can help your body battle the symptoms” or “From your recovery metrics it looks like your body is still doing ok, so some light activity can help relieve the symptoms. Hope you'll feel better tomorrow!” In cases where the timing/duration of the predicted hot flash was not optimal, the messages 1010 may provide suggestions for the user in order to improve their general health. For example, the message 1010 may indicate “If you feel really low on energy, why not try switching to rest mode for today,” or “Since you are having a hot flash, devote today for rest.” For user's whose body signals (e.g., body temperature, heart rate, HRV, and the like) may react to the hot flash, the system 200 may display low activity goals around the hot flash date. In such cases, accurately predicting the hot flash may increase the accuracy and efficiency of the Readiness Score and Activity Scores.

In some cases, the system 200 may be configured to identify relationships between lifestyle choices and outcome of the hot flash. In such cases, the system 200 may be configured to identify the user's triggers for hot flashes and how different lifestyle choices may impact the occurrence and frequency of hot flashes experienced by the user. For example, the messages 1010 displayed to the user 102 via the GUI 1000 of the user device 106 may indicate how a remedial action affects the hot flash. The message 1010 may indicate “Try ten minutes of breathing meditation every night for two weeks and track how your sleep quality changes.” In other examples, the message 1010 may indicate “Try fifteen minutes of walking every day for two weeks and track how menopausal symptoms change.”

In cases where the user 102 dismisses the prompt (e.g., message 1010) on application page, the prompt may disappear, and the user may input an indication of a hot flash via input 1015 at a later time. If the user's hot flash occurs after the predicted hot flash day, the system 200 may display the message 1010 every day during the predicted range of days. If the user's hot flash does not occur during the range of predicted days, then the system 200 may prompt the user to input the hot flash, via input 1015, when the hot flash occurs.

In other examples, if the user's hot flash occurs early (i.e., the hot flash occurs before the predicted hot flash), the user 102 may submit an indication of the hot flash via input 1015. In some cases, estimating the hot flash may be based on receiving the indication of the hot flash. For example, the server of system 200 may receive user input, via input 1015, information associated with the hot flash. In such cases, the system 200 may receive, via the input 1015, supplemental data associated with the physiological data. The supplemental data may include indications of events, subjective attributes, or both. The system 200 may identify the hot flash for the user based on the supplemental data.

Conversely, upon confirming the predicted hot flash, the GUI 1000 may indicate one or more parameters of the hot flash, including a temperature, heart rate, HRV, and the like experienced by the user during the hot flash. In some cases, the application page may include a calendar view that may indicate a current date that the user 102 is viewing application page and a date range including the day when the hot flash is predicted. For example, the date range may encircle the calendar days using a dashed line configuration and the current day may shade-in the calendar day.

In some cases, the user 102 may log symptoms via input 1015. For example, the system 200 may receive user input (e.g., tags) to log symptoms associated with the hot flash (e.g., migraine, pain, perspiration, etc.). In some examples, the system 200 may receive supplemental data such as alcohol input, stress, anxiety, wake-ups in the middle of the night, and the like. In such cases, the system 200 may identify the hot flash based on the supplemental data. The system 200 may recommend tags to the user 102 based on user history and the predicted hot flash. For example, the system 200 may identify that, when the user tags alcohol, caffeine, spicy food, or a combination thereof, or when the user experiences poor sleep, the frequency of their hot flashes may increase. In such cases, the users may learn about relationships between choices and outcomes, and the users may learn their triggers for hot flashes and how different choices may impact the occurrence and frequency of hot flashes.

The GUI 1000 may also include messages 1010 that includes insights, recommendations, and the like associated with the hot flash. The server 110 of system 200 may cause the GUI 1000 of the user device 106 to display a message 1010 associated with the predicted hot flash. The user device 106 may display recommendations and/or information associated with the hot flash via message 1010. As noted previously herein, an accurately predicted hot flash may be beneficial to a user's overall health by providing metrics to the user that may enable the user to understand how behavior changes (e.g., improvements in sleep, exercise, diet, and mood) may help reduce their hot flashes or other perimenopausal or menopausal symptoms. In some implementations, the user device 106 and/or servers 110 may generate alerts 1005 associated with the hot flash that may be displayed to the user via the GUI 1000. In particular, alerts 1005 generated and displayed to the user via the GUI 1000 may be associated with one or more characteristics (e.g., time of day, duration,) of the predicted hot flash.

For example, the alerts 1005 may include a time of day that the future hot flash will occur, a duration between the future hot flash and a previous hot flash, a time interval that the future hot flash is predicted to occur, a request to input symptoms associated with the future hot flash, or a combination thereof. In some cases, the alert 1005 may display a recommendation of how to adjust their lifestyle in the days leading up the predicted hot flash, on the day of the predicted hot flash, and/or in the days after the predicted hot flash. In some examples, the system 200 may recommend a time (e.g., calendar day) for the user 102 to be active or estimate a restorative time following the predicted hot flash.

In some implementations, the system 200 may provide additional insight regarding the user's predicted hot flash. For example, the application pages may indicate one or more physiological parameters (e.g., contributing factors) that resulted in the user's predicted hot flash, such as increased temperature, and the like. In other words, the system 200 may be configured to provide some information or other insights regarding the predicted hot flash. Personalized insights may indicate aspects of collected physiological data (e.g., contributing factors within the physiological data) that were used to generate the predicted hot flash. In some cases, the system 200 may be configured to identify relationships between hot flashes and other menopausal symptoms. For example, the system 200 may be configured to identify where a user is in the reproductive life cycle.

In some implementations, the system 200 may be configured to receive user inputs regarding detected/predicted hot flashes in order to train classifiers (e.g., supervised learning for a machine learning classifier) and improve hot flash prediction techniques. For example, the user device 106 may display a predicted hot flash or range of days indicating a relative likelihood that the user will experience a hot flash. Subsequently, the user 102 may input one or more user inputs, such as an onset of symptoms, a confirmation of the hot flash, and the like. These user inputs may then be input into the classifier to train the classifier. In other words, the user inputs may be used to validate, or confirm, the predicted hot flash.

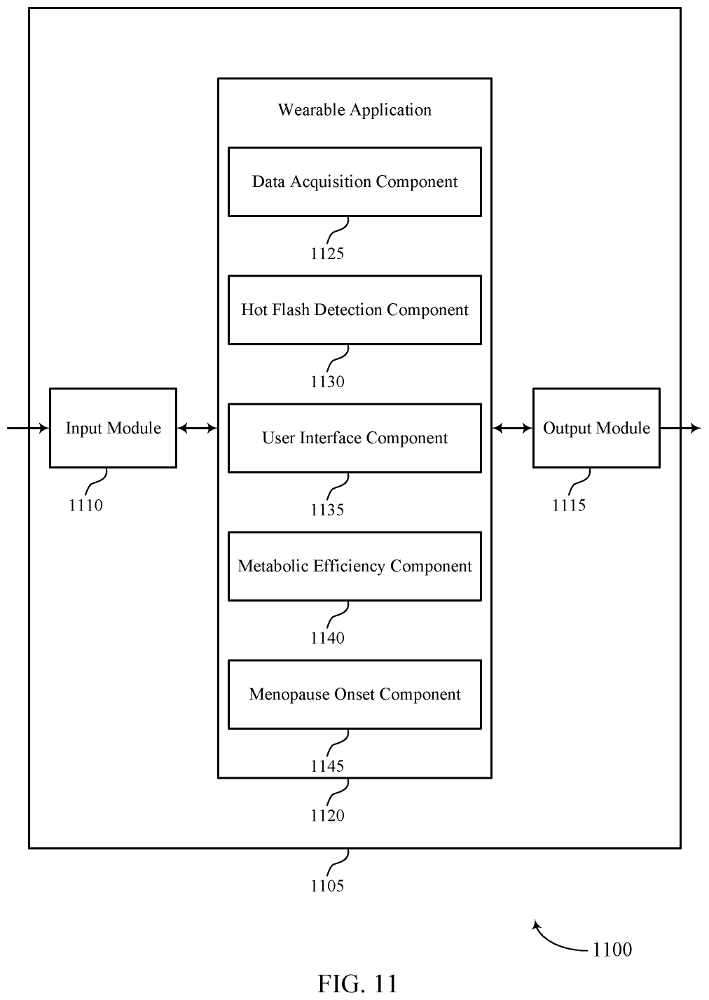

FIG. 11 shows a block diagram 1100 of a device 1105 that supports techniques for menopause and hot flash detection and treatment in accordance with aspects of the present disclosure. The device 1105 may include an input module 1110, an output module 1115, and a wearable application 1120. The device 1105 may also include a processor. Each of these components may be in communication with one another (e.g., via one or more buses).

The input module 1110 may provide a means for receiving information such as packets, user data, control information, or any combination thereof associated with various information channels (e.g., control channels, data channels, information channels related to illness detection techniques). Information may be passed on to other components of the device 1105. The input module 1110 may utilize a single antenna or a set of multiple antennas.

The output module 1115 may provide a means for transmitting signals generated by other components of the device 1105. For example, the output module 1115 may transmit information such as packets, user data, control information, or any combination thereof associated with various information channels (e.g., control channels, data channels, information channels related to illness detection techniques). In some examples, the output module 1115 may be co-located with the input module 1110 in a transceiver module. The output module 1115 may utilize a single antenna or a set of multiple antennas.

For example, the wearable application 1120 may include a data acquisition component 1125, a hot flash detection component 1130, a user interface component 1135, a metabolic efficiency component 1140, a menopause onset component 1145, or any combination thereof. In some examples, the wearable application 1120, or various components thereof, may be configured to perform various operations (e.g., receiving, monitoring, transmitting) using or otherwise in cooperation with the input module 1110, the output module 1115, or both. For example, the wearable application 1120 may receive information from the input module 1110, send information to the output module 1115, or be integrated in combination with the input module 1110, the output module 1115, or both to receive information, transmit information, or perform various other operations as described herein.

The wearable application 1120 may support detecting hot flashes in accordance with examples as disclosed herein. The data acquisition component 1125 may be configured as or otherwise support a means for receiving physiological data associated with a user from a wearable device. The hot flash detection component 1130 may be configured as or otherwise support a means for identifying one or more physiological indications of a hot flash experienced by the user based at least in part on the physiological data satisfying one or more thresholds. The user interface component 1135 may be configured as or otherwise support a means for causing a graphical user interface of a user device to display information associated with the hot flash.

The data acquisition component 1125 may be configured as or otherwise support a means for receiving physiological data associated with a user from a wearable device. The metabolic efficiency component 1140 may be configured as or otherwise support a means for determine a metabolic efficiency metric associated with the user based at least in part on the received physiological data. The menopause onset component 1145 may be configured as or otherwise support a means for determining a menopause metric for the user based at least in part on the metabolic efficiency metric, the menopause metric associated with a relative probability that the user will experience menopausal symptoms. The user interface component 1135 may be configured as or otherwise support a means for causing a graphical user interface of a user device to display one or more messages associated with the menopause metric.

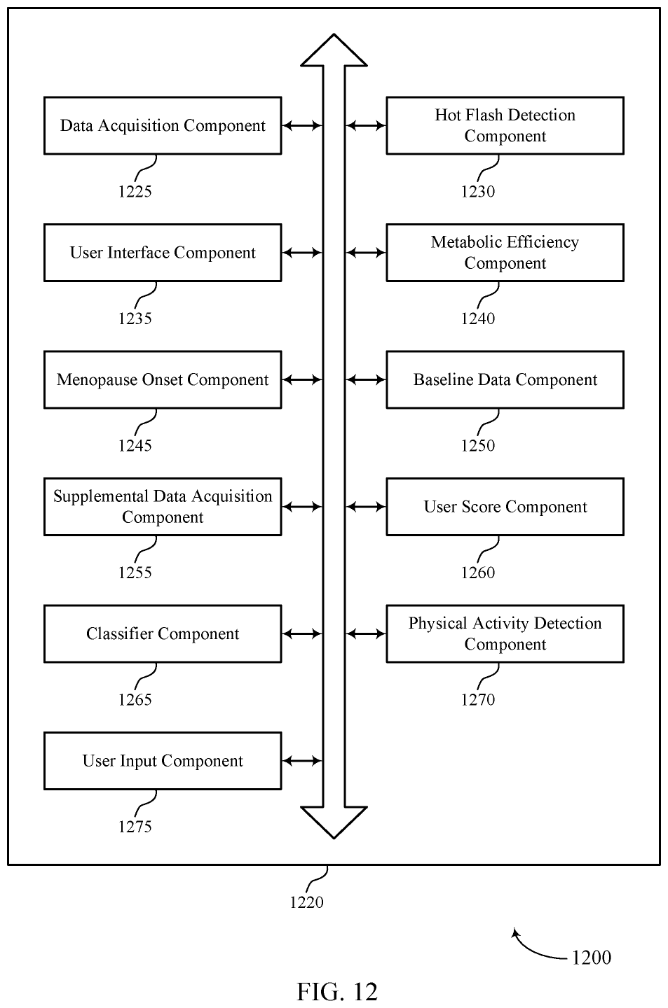

FIG. 12 shows a block diagram 1200 of a wearable application 1220 that supports techniques for menopause and hot flash detection and treatment in accordance with aspects of the present disclosure. The wearable application 1220 may be an example of aspects of a wearable application or a wearable application 1120, or both, as described herein. The wearable application 1220, or various components thereof, may be an example of means for performing various aspects of techniques for menopause and hot flash detection and treatment as described herein. For example, the wearable application 1220 may include a data acquisition component 1225, a hot flash detection component 1230, a user interface component 1235, a metabolic efficiency component 1240, a menopause onset component 1245, a baseline data component 1250, a supplemental data acquisition component 1255, a user score component 1260, a classifier component 1265, a physical activity detection component 1270, a user input component 1275, or any combination thereof. Each of these components may communicate, directly or indirectly, with one another (e.g., via one or more buses).

The wearable application 1220 may support detecting hot flashes in accordance with examples as disclosed herein. The data acquisition component 1225 may be configured as or otherwise support a means for receiving physiological data associated with a user from a wearable device. The hot flash detection component 1230 may be configured as or otherwise support a means for identifying one or more physiological indications of a hot flash experienced by the user based at least in part on the physiological data satisfying one or more thresholds. The user interface component 1235 may be configured as or otherwise support a means for causing a graphical user interface of a user device to display information associated with the hot flash.

In some examples, the data acquisition component 1225 may be configured as or otherwise support a means for receiving additional physiological data associated with the user from the wearable device. In some examples, the baseline data component 1250 may be configured as or otherwise support a means for determining baseline physiological data for the user based at least in part on the additional physiological data, wherein the one or more thresholds are based on the baseline physiological data for the user.

In some examples, the supplemental data acquisition component 1255 may be configured as or otherwise support a means for receiving, via the user device, supplemental data associated with the physiological data, the supplemental data comprising indications of events, subjective attributes, or both. In some examples, the hot flash detection component 1230 may be configured as or otherwise support a means for identifying the one or more physiological indications of the hot flash based at least in part on the supplemental data. In some examples, the supplemental data comprises an indication of one or more hot flashes, one or more tags associated with the physiological data, or both.

In some examples, the hot flash detection component 1230 may be configured as or otherwise support a means for identifying one or more hot flash triggers for the user based at least in part on identifying the hot flash, the supplemental data, or both. In some examples, the user interface component 1235 may be configured as or otherwise support a means for causing the graphical user interface of the user device to display an indication of the one or more hot flash triggers.

In some examples, the data acquisition component 1225 may be configured as or otherwise support a means for receiving additional physiological data associated with the user from the wearable device. In some examples, the hot flash detection component 1230 may be configured as or otherwise support a means for determining a hot flash risk metric for the user based at least in part on the additional physiological data, the hot flash risk metric associated with a relative probability that the user will experience a potential hot flash. In some examples, the user interface component 1235 may be configured as or otherwise support a means for causing the graphical user interface of the user device to display an indication of the potential hot flash based at least in part on the hot flash risk metric satisfying a hot flash prediction threshold.

In some examples, the user score component 1260 may be configured as or otherwise support a means for adjusting one or more scores for the user based at least in part on the identified hot flash, wherein the one or more scores comprise a Sleep Score, a Readiness Score, or both.

In some examples, to support identifying the one or more physiological indications of the hot flash, the data acquisition component 1225 may be configured as or otherwise support a means for identifying a first change in the temperature data within a first time interval is greater than a temperature change threshold. In some examples, to support identifying the one or more physiological indications of the hot flash, the data acquisition component 1225 may be configured as or otherwise support a means for identifying a second change in the heart rate data within the first time interval is greater than a heart rate change threshold.

In some examples, the classifier component 1265 may be configured as or otherwise support a means for inputting the received physiological data into a classifier, wherein the classifier is configured to identify the one or more physiological indications of the hot flash based at least in part on the received physiological data.

In some examples, the user input component 1275 may be configured as or otherwise support a means for receiving, via the user device, a user input that confirms or denies the identified hot flash. In some examples, the classifier component 1265 may be configured as or otherwise support a means for inputting the user input into the classifier to train the classifier for hot flash detection.

In some examples, the physiological data comprises temperature data, heart rate data, respiratory rate data, galvanic skin response data, or any combination thereof. In some examples, the wearable device comprises a wearable ring device. In some examples, the wearable device collects the physiological data from the user based on arterial blood flow.

In some examples, the data acquisition component 1225 may be configured as or otherwise support a means for receiving physiological data associated with a user from a wearable device. The metabolic efficiency component 1240 may be configured as or otherwise support a means for determine a metabolic efficiency metric associated with the user based at least in part on the received physiological data. The menopause onset component 1245 may be configured as or otherwise support a means for determining a menopause metric for the user based at least in part on the metabolic efficiency metric, the menopause metric associated with a relative probability that the user will experience menopausal symptoms. In some examples, the user interface component 1235 may be configured as or otherwise support a means for causing a graphical user interface of a user device to display one or more messages associated with the menopause metric.

In some examples, the metabolic efficiency component 1240 may be configured as or otherwise support a means for determining a change in the metabolic efficiency metric associated with the user based at least in part on identifying the metabolic efficiency metric, wherein determining the menopause metric is based at least in part on the change in the metabolic efficiency metric.

In some examples, the data acquisition component 1225 may be configured as or otherwise support a means for determining that the sleep data deviates from a baseline sleep threshold for the user, wherein determining the menopause metric is based at least in part on determining that the sleep data deviates from the baseline sleep threshold for the user.

In some examples, the physical activity detection component 1270 may be configured as or otherwise support a means for identifying a time interval that the user is engaged in physical activity based at least in part on the physiological data. In some examples, the data acquisition component 1225 may be configured as or otherwise support a means for determining a first rate of change associated with metabolic equivalent (MET) data for the user during the time interval based at least in part on the physiological data. In some examples, the data acquisition component 1225 may be configured as or otherwise support a means for determining a second rate of change associated with temperature data for the user during the time interval based at least in part on the physiological data, wherein determining the metabolic efficiency metric is based at least in part on the first rate of change, the second rate of change, or both.

In some examples, the supplemental data acquisition component 1255 may be configured as or otherwise support a means for receiving, via the user device, supplemental data associated with the physiological data, the supplemental data comprising indications of events, subjective attributes, or both. In some examples, the menopause onset component 1245 may be configured as or otherwise support a means for determining the menopause metric for the user based at least in part on the supplemental data.

In some examples, the one or more messages comprise a prediction of menopausal symptoms, exercise recommendations, insights associated with the physiological activity, or any combination thereof. In some examples, the metabolic efficiency metric is indicative of a relative efficiency that cells of the user produce cellular energy. In some examples, the metabolic efficiency metric is associated with a thermoregulatory ability of the user to regulate internal body temperature.

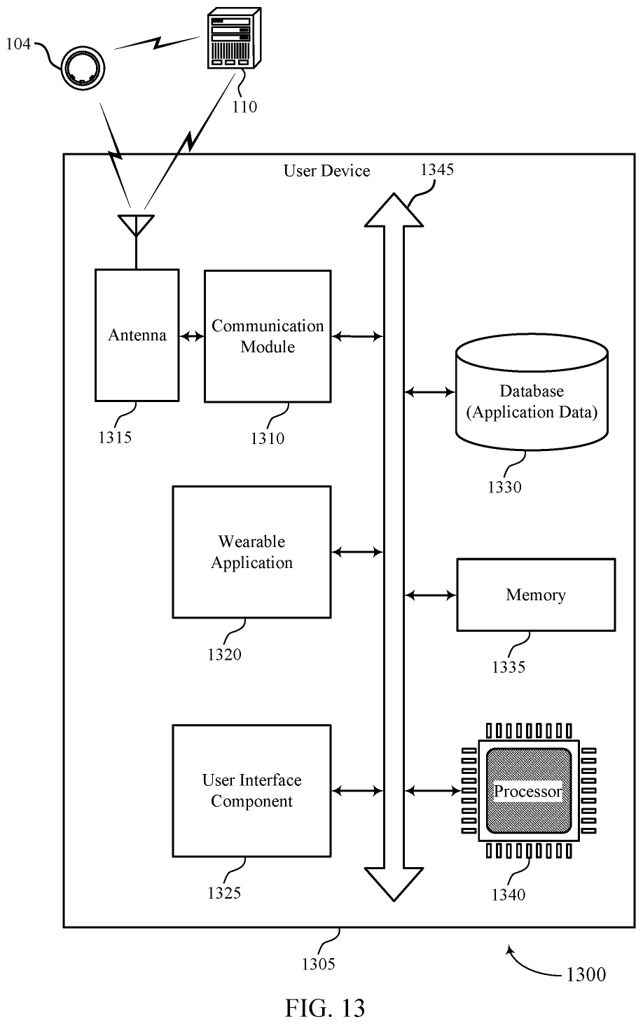

FIG. 13 shows a diagram of a system 1300 including a device 1305 that supports techniques for menopause and hot flash detection and treatment in accordance with aspects of the present disclosure. The device 1305 may be an example of or include the components of a device 1105 as described herein. The device 1305 may include components for bi-directional communications with a ring 104 and a server 110 including components for transmitting and receiving communications, such as a wearable application 1320, a communication module 1310, an antenna 1315, a user interface component 1325, a database (application data) 1330, a memory 1335, and a processor 1340. These components may be in electronic communication or otherwise coupled (e.g., operatively, communicatively, functionally, electronically, electrically) via one or more buses (e.g., a bus 1345).

The communication module 1310 may manage input and output signals for the device 1305 via the antenna 1315. The communication module 1310 may include an example of the communication module 220-b of the user device 106 shown and described in FIG. 2. In this regard, the communication module 1310 may manage communications with the ring 104 and the server 110, as illustrated in FIG. 2. The communication module 1310 may also manage peripherals not integrated into the device 1305. In some cases, the communication module 1310 may represent a physical connection or port to an external peripheral. In some cases, the communication module 1310 may utilize an operating system such as iOS®, ANDROID®, MS-DOS®, MS-WINDOWS®, OS/2®, UNIX®, LINUX®, or another known operating system. In other cases, the communication module 1310 may represent or interact with a wearable device (e.g., ring 104), modem, a keyboard, a mouse, a touchscreen, or a similar device. In some cases, the communication module 1310 may be implemented as part of the processor 1340. In some examples, a user may interact with the device 1305 via the communication module 1310, user interface component 1325, or via hardware components controlled by the communication module 1310.

In some cases, the device 1305 may include a single antenna 1315. However, in some other cases, the device 1305 may have more than one antenna 1315, that may be capable of concurrently transmitting or receiving multiple wireless transmissions. The communication module 1310 may communicate bi-directionally, via the one or more antennas 1315, wired, or wireless links as described herein. For example, the communication module 1310 may represent a wireless transceiver and may communicate bi-directionally with another wireless transceiver. The communication module 1310 may also include a modem to modulate the packets, to provide the modulated packets to one or more antennas 1315 for transmission, and to demodulate packets received from the one or more antennas 1315.

The user interface component 1325 may manage data storage and processing in a database 1330. In some cases, a user may interact with the user interface component 1325. In other cases, the user interface component 1325 may operate automatically without user interaction. The database 1330 may be an example of a single database, a distributed database, multiple distributed databases, a data store, a data lake, or an emergency backup database.

The memory 1335 may include RAM and ROM. The memory 1335 may store computer-readable, computer-executable software including instructions that, when executed, cause the processor 1340 to perform various functions described herein. In some cases, the memory 1335 may contain, among other things, a BIOS that may control basic hardware or software operation such as the interaction with peripheral components or devices.

The processor 1340 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 1340 may be configured to operate a memory array using a memory controller. In other cases, a memory controller may be integrated into the processor 1340. The processor 1340 may be configured to execute computer-readable instructions stored in a memory 1335 to perform various functions (e.g., functions or tasks supporting a method and system for sleep staging algorithms).

The wearable application 1320 may support detecting hot flashes in accordance with examples as disclosed herein. For example, the wearable application 1320 may be configured as or otherwise support a means for receiving physiological data associated with a user from a wearable device. The wearable application 1320 may be configured as or otherwise support a means for identifying one or more physiological indications of a hot flash experienced by the user based at least in part on the physiological data satisfying one or more thresholds. The wearable application 1320 may be configured as or otherwise support a means for causing a graphical user interface of a user device to display information associated with the hot flash.

For example, the wearable application 1320 may be configured as or otherwise support a means for receiving physiological data associated with a user from a wearable device. The wearable application 1320 may be configured as or otherwise support a means for determining a metabolic efficiency metric associated with the user based at least in part on the received physiological data. The wearable application 1320 may be configured as or otherwise support a means for determining a menopause metric for the user based at least in part on the metabolic efficiency metric, the menopause metric associated with a relative probability that the user will experience menopausal symptoms. The wearable application 1320 may be configured as or otherwise support a means for causing a graphical user interface of a user device to display one or more messages associated with the menopause metric.

By including or configuring the wearable application 1320 in accordance with examples as described herein, the device 1305 may support techniques for In particular, techniques described herein may be used to predict menopausal symptoms (e.g., hot flashes) for a given user, that may be used to generate more accurate predictions for the user. By providing a user with a more comprehensive prediction of their menopausal symptom onsets, techniques described herein may enable the user to effectively adjust their lifestyle patterns, that may improve the overall health for the user.

The wearable application 1320 may include an application (e.g., “app”), program, software, or other component that is configured to facilitate communications with a ring 104, server 110, other user devices 106, and the like. For example, the wearable application 1320 may include an application executable on a user device 106 that is configured to receive data (e.g., physiological data) from a ring 104, perform processing operations on the received data, transmit and receive data with the servers 110, and cause presentation of data to a user 102.

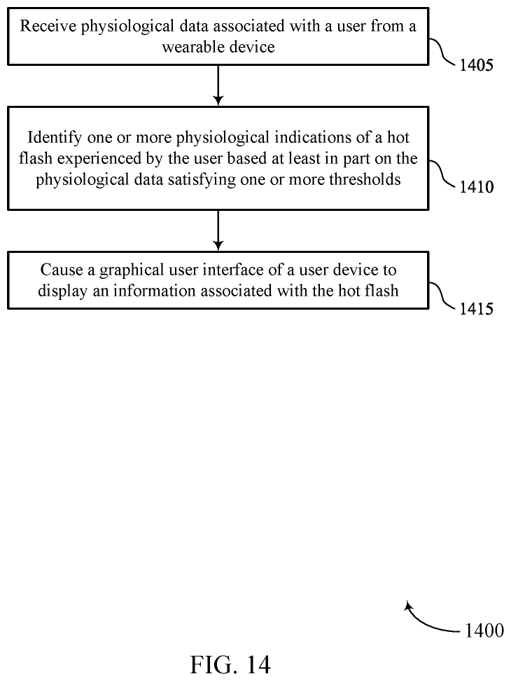

FIG. 14 shows a flowchart illustrating a method 1400 that supports techniques for menopause and hot flash detection and treatment in accordance with aspects of the present disclosure. The operations of the method 1400 may be implemented by a user device or its components as described herein. For example, the operations of the method 1400 may be performed by a user device as described with reference to FIGS. 1 through 13. In some examples, a user device may execute a set of instructions to control the functional elements of the user device to perform the described functions. Additionally, or alternatively, the user device may perform aspects of the described functions using special-purpose hardware.

At 1405, the method may include receiving physiological data associated with a user from a wearable device. The operations of 1405 may be performed in accordance with examples as disclosed herein. In some examples, aspects of the operations of 1405 may be performed by a data acquisition component 1225 as described with reference to FIG. 12.

At 1410, the method may include identifying one or more physiological indications of a hot flash experienced by the user based at least in part on the physiological data satisfying one or more thresholds. The operations of 1410 may be performed in accordance with examples as disclosed herein. In some examples, aspects of the operations of 1410 may be performed by a hot flash detection component 1230 as described with reference to FIG. 12.

At 1415, the method may include causing a graphical user interface of a user device to display information associated with the hot flash. The operations of 1415 may be performed in accordance with examples as disclosed herein. In some examples, aspects of the operations of 1415 may be performed by a user interface component 1235 as described with reference to FIG. 12.

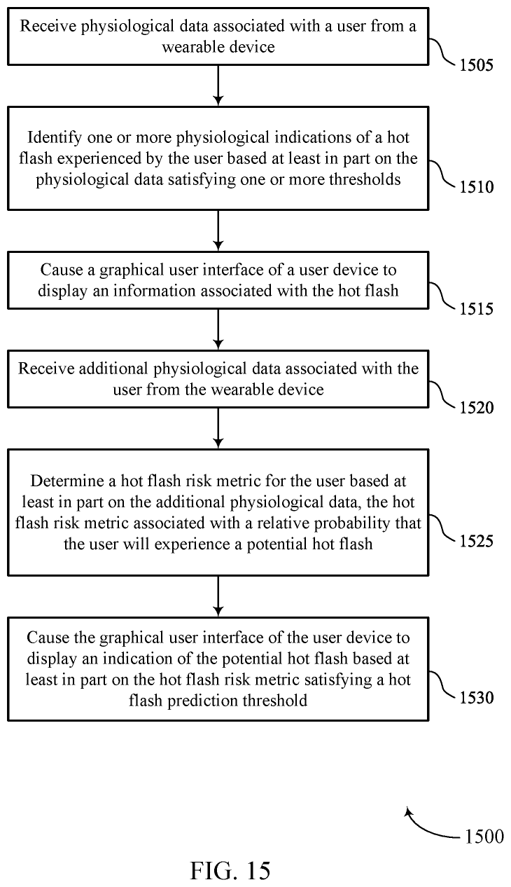

FIG. 15 shows a flowchart illustrating a method 1500 that supports techniques for menopause and hot flash detection and treatment in accordance with aspects of the present disclosure. The operations of the method 1500 may be implemented by a user device or its components as described herein. For example, the operations of the method 1500 may be performed by a user device as described with reference to FIGS. 1 through 13. In some examples, a user device may execute a set of instructions to control the functional elements of the user device to perform the described functions. Additionally, or alternatively, the user device may perform aspects of the described functions using special-purpose hardware.

At 1505, the method may include receiving physiological data associated with a user from a wearable device. The operations of 1505 may be performed in accordance with examples as disclosed herein. In some examples, aspects of the operations of 1505 may be performed by a data acquisition component 1225 as described with reference to FIG. 12.

At 1510, the method may include identifying one or more physiological indications of a hot flash experienced by the user based at least in part on the physiological data satisfying one or more thresholds. The operations of 1510 may be performed in accordance with examples as disclosed herein. In some examples, aspects of the operations of 1510 may be performed by a hot flash detection component 1230 as described with reference to FIG. 12.

At 1515, the method may include causing a graphical user interface of a user device to display information associated with the hot flash. The operations of 1515 may be performed in accordance with examples as disclosed herein. In some examples, aspects of the operations of 1515 may be performed by a user interface component 1235 as described with reference to FIG. 12.

At 1520, the method may include receiving additional physiological data associated with the user from the wearable device. The operations of 1520 may be performed in accordance with examples as disclosed herein. In some examples, aspects of the operations of 1520 may be performed by a data acquisition component 1225 as described with reference to FIG. 12.

At 1525, the method may include determining a hot flash risk metric for the user based at least in part on the additional physiological data, the hot flash risk metric associated with a relative probability that the user will experience a potential hot flash. The operations of 1525 may be performed in accordance with examples as disclosed herein. In some examples, aspects of the operations of 1525 may be performed by a hot flash detection component 1230 as described with reference to FIG. 12.

At 1530, the method may include causing the graphical user interface of the user device to display an indication of the potential hot flash based at least in part on the hot flash risk metric satisfying a hot flash prediction threshold. The operations of 1530 may be performed in accordance with examples as disclosed herein. In some examples, aspects of the operations of 1530 may be performed by a user interface component 1235 as described with reference to FIG. 12.

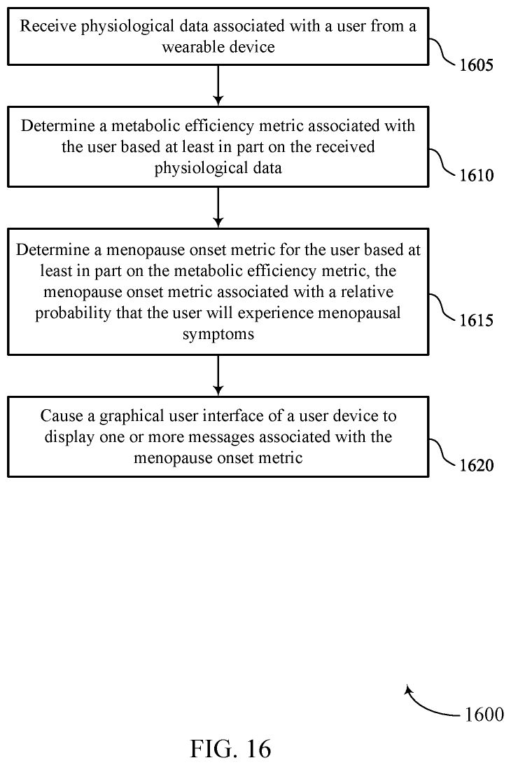

FIG. 16 shows a flowchart illustrating a method 1600 that supports techniques for menopause and hot flash detection and treatment in accordance with aspects of the present disclosure. The operations of the method 1600 may be implemented by a user device or its components as described herein. For example, the operations of the method 1600 may be performed by a user device as described with reference to FIGS. 1 through 13. In some examples, a user device may execute a set of instructions to control the functional elements of the user device to perform the described functions. Additionally, or alternatively, the user device may perform aspects of the described functions using special-purpose hardware.

At 1605, the method may include receiving physiological data associated with a user from a wearable device. The operations of 1605 may be performed in accordance with examples as disclosed herein. In some examples, aspects of the operations of 1605 may be performed by a data acquisition component 1225 as described with reference to FIG. 12.

At 1610, the method may include determine a metabolic efficiency metric associated with the user based at least in part on the received physiological data. The operations of 1610 may be performed in accordance with examples as disclosed herein. In some examples, aspects of the operations of 1610 may be performed by a metabolic efficiency component 1240 as described with reference to FIG. 12.

At 1615, the method may include determining a menopause metric for the user based at least in part on the metabolic efficiency metric, the menopause metric associated with a relative probability that the user will experience menopausal symptoms. The operations of 1615 may be performed in accordance with examples as disclosed herein. In some examples, aspects of the operations of 1615 may be performed by a menopause onset component 1245 as described with reference to FIG. 12.

At 1620, the method may include causing a graphical user interface of a user device to display one or more messages associated with the menopause metric. The operations of 1620 may be performed in accordance with examples as disclosed herein. In some examples, aspects of the operations of 1620 may be performed by a user interface component 1235 as described with reference to FIG. 12.

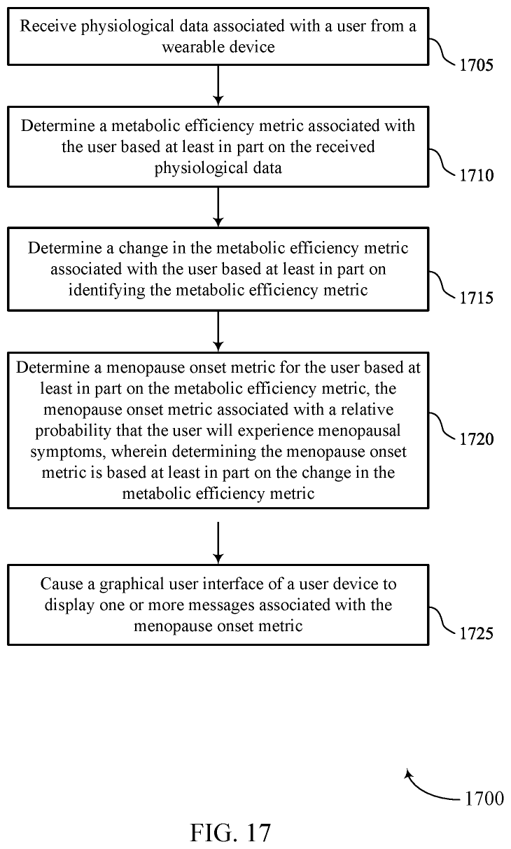

FIG. 17 shows a flowchart illustrating a method 1700 that supports techniques for menopause and hot flash detection and treatment in accordance with aspects of the present disclosure. The operations of the method 1700 may be implemented by a user device or its components as described herein. For example, the operations of the method 1700 may be performed by a user device as described with reference to FIGS. 1 through 13. In some examples, a user device may execute a set of instructions to control the functional elements of the user device to perform the described functions. Additionally, or alternatively, the user device may perform aspects of the described functions using special-purpose hardware.

At 1705, the method may include receiving physiological data associated with a user from a wearable device. The operations of 1705 may be performed in accordance with examples as disclosed herein. In some examples, aspects of the operations of 1705 may be performed by a data acquisition component 1225 as described with reference to FIG. 12.

At 1710, the method may include determine a metabolic efficiency metric associated with the user based at least in part on the received physiological data. The operations of 1710 may be performed in accordance with examples as disclosed herein. In some examples, aspects of the operations of 1710 may be performed by a metabolic efficiency component 1240 as described with reference to FIG. 12.

At 1715, the method may include determining a change in the metabolic efficiency metric associated with the user based at least in part on identifying the metabolic efficiency metric. The operations of 1715 may be performed in accordance with examples as disclosed herein. In some examples, aspects of the operations of 1715 may be performed by a metabolic efficiency component 1240 as described with reference to FIG. 12.

At 1720, the method may include determining a menopause metric for the user based at least in part on the metabolic efficiency metric, the menopause metric associated with a relative probability that the user will experience menopausal symptoms, wherein determining the menopause metric is based at least in part on the change in the metabolic efficiency metric. The operations of 1720 may be performed in accordance with examples as disclosed herein. In some examples, aspects of the operations of 1720 may be performed by a menopause onset component 1245 as described with reference to FIG. 12.

At 1725, the method may include causing a graphical user interface of a user device to display one or more messages associated with the menopause metric. The operations of 1725 may be performed in accordance with examples as disclosed herein. In some examples, aspects of the operations of 1725 may be performed by a user interface component 1235 as described with reference to FIG. 12.

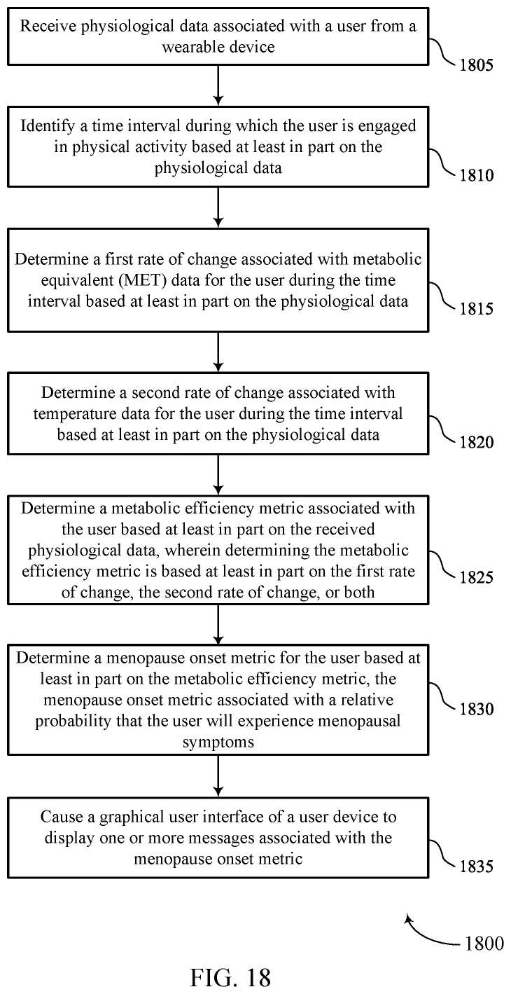

FIG. 18 shows a flowchart illustrating a method 1800 that supports techniques for menopause and hot flash detection and treatment in accordance with aspects of the present disclosure. The operations of the method 1800 may be implemented by a user device or its components as described herein. For example, the operations of the method 1800 may be performed by a user device as described with reference to FIGS. 1 through 13. In some examples, a user device may execute a set of instructions to control the functional elements of the user device to perform the described functions. Additionally, or alternatively, the user device may perform aspects of the described functions using special-purpose hardware.

At 1805, the method may include receiving physiological data associated with a user from a wearable device. The operations of 1805 may be performed in accordance with examples as disclosed herein. In some examples, aspects of the operations of 1805 may be performed by a data acquisition component 1225 as described with reference to FIG. 12.

At 1810, the method may include identifying a time interval that the user is engaged in physical activity based at least in part on the physiological data. The operations of 1810 may be performed in accordance with examples as disclosed herein. In some examples, aspects of the operations of 1810 may be performed by a physical activity detection component 1270 as described with reference to FIG. 12.

At 1815, the method may include determining a first rate of change associated with metabolic equivalent (MET) data for the user during the time interval based at least in part on the physiological data. The operations of 1815 may be performed in accordance with examples as disclosed herein. In some examples, aspects of the operations of 1815 may be performed by a data acquisition component 1225 as described with reference to FIG. 12.

At 1820, the method may include determining a second rate of change associated with temperature data for the user during the time interval based at least in part on the physiological data. The operations of 1820 may be performed in accordance with examples as disclosed herein. In some examples, aspects of the operations of 1820 may be performed by a data acquisition component 1225 as described with reference to FIG. 12.

At 1825, the method may include determine a metabolic efficiency metric associated with the user based at least in part on the received physiological data, wherein determining the metabolic efficiency metric is based at least in part on the first rate of change, the second rate of change, or both. The operations of 1825 may be performed in accordance with examples as disclosed herein. In some examples, aspects of the operations of 1825 may be performed by a metabolic efficiency component 1240 as described with reference to FIG. 12.

At 1830, the method may include determining a menopause metric for the user based at least in part on the metabolic efficiency metric, the menopause metric associated with a relative probability that the user will experience menopausal symptoms. The operations of 1830 may be performed in accordance with examples as disclosed herein. In some examples, aspects of the operations of 1830 may be performed by a menopause onset component 1245 as described with reference to FIG. 12.

At 1835, the method may include causing a graphical user interface of a user device to display one or more messages associated with the menopause metric. The operations of 1835 may be performed in accordance with examples as disclosed herein. In some examples, aspects of the operations of 1835 may be performed by a user interface component 1235 as described with reference to FIG. 12.

It should be noted that the methods described above describe possible implementations, and that the operations and the steps may be rearranged or otherwise modified and that other implementations are possible. Furthermore, aspects from two or more of the methods may be combined.

A method for detecting hot flashes is described. The method may include receiving physiological data associated with a user from a wearable device, identifying one or more physiological indications of a hot flash experienced by the user based at least in part on the physiological data satisfying one or more thresholds, and causing a graphical user interface of a user device to display information associated with the hot flash.

An apparatus for detecting hot flashes is described. The apparatus may include a processor, memory coupled with the processor, and instructions stored in the memory. The instructions may be executable by the processor to cause the apparatus to receive physiological data associated with a user from a wearable device, identify one or more physiological indications of a hot flash experienced by the user based at least in part on the physiological data satisfying one or more thresholds, and cause a graphical user interface of a user device to display information associated with the hot flash.

Another apparatus for detecting hot flashes is described. The apparatus may include means for receiving physiological data associated with a user from a wearable device, means for identifying one or more physiological indications of a hot flash experienced by the user based at least in part on the physiological data satisfying one or more thresholds, and means for causing a graphical user interface of a user device to display information associated with the hot flash.

A non-transitory computer-readable medium storing code for detecting hot flashes is described. The code may include instructions executable by a processor to receive physiological data associated with a user from a wearable device, identify one or more physiological indications of a hot flash experienced by the user based at least in part on the physiological data satisfying one or more thresholds, and cause a graphical user interface of a user device to display information associated with the hot flash.

Some examples of the method, apparatuses, and non-transitory computer-readable medium described herein may further include operations, features, means, or instructions for receiving additional physiological data associated with the user from the wearable device and determining baseline physiological data for the user based at least in part on the additional physiological data, wherein the one or more thresholds may be based on the baseline physiological data for the user.

Some examples of the method, apparatuses, and non-transitory computer-readable medium described herein may further include operations, features, means, or instructions for receiving, via the user device, supplemental data associated with the physiological data, the supplemental data comprising indications of events, subjective attributes, or both and identifying the one or more physiological indications of the hot flash based at least in part on the supplemental data.

In some examples of the method, apparatuses, and non-transitory computer-readable medium described herein, the supplemental data comprises an indication of one or more hot flashes, one or more tags associated with the physiological data, or both.

Some examples of the method, apparatuses, and non-transitory computer-readable medium described herein may further include operations, features, means, or instructions for identifying one or more hot flash triggers for the user based at least in part on identifying the hot flash, the supplemental data, or both and causing the graphical user interface of the user device to display an indication of the one or more hot flash triggers.

Some examples of the method, apparatuses, and non-transitory computer-readable medium described herein may further include operations, features, means, or instructions for receiving additional physiological data associated with the user from the wearable device, determining a hot flash risk metric for the user based at least in part on the additional physiological data, the hot flash risk metric associated with a relative probability that the user will experience a potential hot flash, and causing the graphical user interface of the user device to display an indication of the potential hot flash based at least in part on the hot flash risk metric satisfying a hot flash prediction threshold.

Some examples of the method, apparatuses, and non-transitory computer-readable medium described herein may further include operations, features, means, or instructions for adjusting one or more scores for the user based at least in part on the identified hot flash, wherein the one or more scores comprise a Sleep Score, a Readiness Score, or both.

In some examples of the method, apparatuses, and non-transitory computer-readable medium described herein, identifying the one or more physiological indications of the hot flash may include operations, features, means, or instructions for identifying a first change in the temperature data within a first time interval may be greater than a temperature change threshold and identifying a second change in the heart rate data within the first time interval may be greater than a heart rate change threshold.

Some examples of the method, apparatuses, and non-transitory computer-readable medium described herein may further include operations, features, means, or instructions for inputting the received physiological data into a classifier, wherein the classifier may be configured to identify the one or more physiological indications of the hot flash based at least in part on the received physiological data.

Some examples of the method, apparatuses, and non-transitory computer-readable medium described herein may further include operations, features, means, or instructions for receiving, via the user device, a user input that confirms or denies the identified hot flash and inputting the user input into the classifier to train the classifier for hot flash detection.

In some examples of the method, apparatuses, and non-transitory computer-readable medium described herein, the physiological data comprises temperature data, heart rate data, respiratory rate data, galvanic skin response data, or any combination thereof.

In some examples of the method, apparatuses, and non-transitory computer-readable medium described herein, the wearable device comprises a wearable ring device.

In some examples of the method, apparatuses, and non-transitory computer-readable medium described herein, the wearable device collects the physiological data from the user based on arterial blood flow.

A method is described. The method may include receiving physiological data associated with a user from a wearable device, determine a metabolic efficiency metric associated with the user based at least in part on the received physiological data, determining a menopause metric for the user based at least in part on the metabolic efficiency metric, the menopause metric associated with a relative probability that the user will experience menopausal symptoms, and causing a graphical user interface of a user device to display one or more messages associated with the menopause metric.

An apparatus is described. The apparatus may include a processor, memory coupled with the processor, and instructions stored in the memory. The instructions may be executable by the processor to cause the apparatus to receive physiological data associated with a user from a wearable device, determine a metabolic efficiency metric associated with the user based at least in part on the received physiological data, determine a menopause metric for the user based at least in part on the metabolic efficiency metric, the menopause metric associated with a relative probability that the user will experience menopausal symptoms, and cause a graphical user interface of a user device to display one or more messages associated with the menopause metric.

Another apparatus is described. The apparatus may include means for receiving physiological data associated with a user from a wearable device, means for determine a metabolic efficiency metric associated with the user based at least in part on the received physiological data, means for determining a menopause metric for the user based at least in part on the metabolic efficiency metric, the menopause metric associated with a relative probability that the user will experience menopausal symptoms, and means for causing a graphical user interface of a user device to display one or more messages associated with the menopause metric.

A non-transitory computer-readable medium storing code is described. The code may include instructions executable by a processor to receive physiological data associated with a user from a wearable device, determine a metabolic efficiency metric associated with the user based at least in part on the received physiological data, determine a menopause metric for the user based at least in part on the metabolic efficiency metric, the menopause metric associated with a relative probability that the user will experience menopausal symptoms, and cause a graphical user interface of a user device to display one or more messages associated with the menopause metric.

Some examples of the method, apparatuses, and non-transitory computer-readable medium described herein may further include operations, features, means, or instructions for determining a change in the metabolic efficiency metric associated with the user based at least in part on identifying the metabolic efficiency metric, wherein determining the menopause metric may be based at least in part on the change in the metabolic efficiency metric.

Some examples of the method, apparatuses, and non-transitory computer-readable medium described herein may further include operations, features, means, or instructions for determining that the sleep data deviates from a baseline sleep threshold for the user, wherein determining the menopause metric is based at least in part on determining that the sleep data deviates from the baseline sleep threshold for the user.

Some examples of the method, apparatuses, and non-transitory computer-readable medium described herein may further include operations, features, means, or instructions for identifying a time interval that the user may be engaged in physical activity based at least in part on the physiological data, determining a first rate of change associated with metabolic equivalent (MET) data for the user during the time interval based at least in part on the physiological data, and determining a second rate of change associated with temperature data for the user during the time interval based at least in part on the physiological data, wherein determining the metabolic efficiency metric may be based at least in part on the first rate of change, the second rate of change, or both.

Some examples of the method, apparatuses, and non-transitory computer-readable medium described herein may further include operations, features, means, or instructions for receiving, via the user device, supplemental data associated with the physiological data, the supplemental data comprising indications of events, subjective attributes, or both and determining the menopause metric for the user based at least in part on the supplemental data.

In some examples of the method, apparatuses, and non-transitory computer-readable medium described herein, the one or more messages comprise a prediction of menopausal symptoms, exercise recommendations, insights associated with the physiological activity, or any combination thereof.

In some examples of the method, apparatuses, and non-transitory computer-readable medium described herein, the metabolic efficiency metric may be indicative of a relative efficiency that cells of the user produce cellular energy.

In some examples of the method, apparatuses, and non-transitory computer-readable medium described herein, the metabolic efficiency metric may be associated with a thermoregulatory ability of the user to regulate internal body temperature.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for detecting hot flashes, comprising:
receiving physiological data associated with a user from a wearable device;
identifying one or more physiological indications of a hot flash experienced by the user based at least in part on the physiological data satisfying one or more thresholds; and
causing a graphical user interface of a user device to display information associated with the hot flash.

2. The method of claim 1, further comprising:
receiving additional physiological data associated with the user from the wearable device;
determining baseline physiological data for the user based at least in part on the additional physiological data, wherein the one or more thresholds are based on the baseline physiological data for the user.

3. The method of claim 1, further comprising:
receiving, via the user device, supplemental data associated with the physiological data, the supplemental data comprising indications of events, subjective attributes, or both; and
identifying the one or more physiological indications of the hot flash based at least in part on the supplemental data.

4. The method of claim 3, wherein the supplemental data comprises an indication of one or more hot flashes, one or more tags associated with the physiological data, or both.

5. The method of claim 3, further comprising:
identifying one or more hot flash triggers for the user based at least in part on identifying the hot flash, the supplemental data, or both; and
causing the graphical user interface of the user device to display an indication of the one or more hot flash triggers.

6. The method of claim 1, further comprising:
receiving additional physiological data associated with the user from the wearable device;
determining a hot flash risk metric for the user based at least in part on the additional physiological data, the hot flash risk metric associated with a relative probability that the user will experience a potential hot flash; and
causing the graphical user interface of the user device to display an indication of the potential hot flash based at least in part on the hot flash risk metric satisfying a hot flash prediction threshold.

7. The method of claim 1, further comprising:
adjusting one or more scores for the user based at least in part on the identified hot flash, wherein the one or more scores comprise a Sleep Score, a Readiness Score, or both.

8. The method of claim 1, wherein the physiological data comprises at least temperature data and heart rate data, wherein identifying the one or more physiological indications of the hot flash comprises:
identifying a first change in the temperature data within a first time interval is greater than a temperature change threshold; and
identifying a second change in the heart rate data within the first time interval is greater than a heart rate change threshold.

9. The method of claim 1, further comprising:
inputting the received physiological data into a classifier, wherein the classifier is configured to identify the one or more physiological indications of the hot flash based at least in part on the received physiological data.

10. The method of claim 9, further comprising:
receiving, via the user device, a user input that confirms or denies the identified hot flash; and
inputting the user input into the classifier to train the classifier for hot flash detection.

11. The method of claim 1, wherein the physiological data comprises temperature data, heart rate data, respiratory rate data, galvanic skin response data, or any combination thereof.

12. The method of claim 1, wherein the wearable device comprises a wearable ring device.

13. The method of claim 1, wherein the wearable device collects the physiological data from the user based on arterial blood flow.

14. A method for menopause prediction, comprising:
receiving physiological data associated with a user from a wearable device;
determining a metabolic efficiency metric associated with the user based at least in part on the received physiological data;
determining a menopause metric for the user based at least in part on the metabolic efficiency metric, the menopause metric associated with a relative probability that the user will experience menopausal symptoms; and
causing a graphical user interface of a user device to display one or more messages associated with the menopause metric.

15. The method of claim 14, further comprising:
determining a change in the metabolic efficiency metric associated with the user based at least in part on identifying the metabolic efficiency metric, wherein determining the menopause metric is based at least in part on the change in the metabolic efficiency metric.

16. The method of claim 14, wherein the physiological data further comprises sleep data, the method further comprising:
determining that the sleep data deviates from a baseline sleep threshold for the user, wherein determining the menopause metric is based at least in part on determining that the sleep data deviates from the baseline sleep threshold for the user.

17. The method of claim 14, further comprising:
identifying a time interval that the user is engaged in physical activity based at least in part on the physiological data;
determining a first rate of change associated with metabolic equivalent (MET) data for the user during the time interval based at least in part on the physiological data; and
determining a second rate of change associated with temperature data for the user during the time interval based at least in part on the physiological data, wherein determining the metabolic efficiency metric is based at least in part on the first rate of change, the second rate of change, or both.

18. The method of claim 14, further comprising:
receiving, via the user device, supplemental data associated with the physiological data, the supplemental data comprising indications of events, subjective attributes, or both; and
determining the menopause metric for the user based at least in part on the supplemental data.

19. The method of claim 14, wherein the one or more messages comprise a prediction of menopausal symptoms, exercise recommendations, insights associated with the physiological activity, or any combination thereof.

20. The method of claim 14, wherein the metabolic efficiency metric is indicative of a relative efficiency that cells of the user produce cellular energy.
